SEQUENCE LISTING (81 sequences)

SEQ ID NO: 1 - protein, 36 aa, Artificial Sequence
  C1 Activation Loop Consensus Sequence
  MOD_RES (2)..(11): Any amino acid
  SITE (2)..(11): This region may encompass 1-10 residues
  MOD_RES (13)..(13): Asp or Glu
  MOD_RES (16)..(30): Any amino acid
  SITE (16)..(35): This region may encompass 4-20 residues
CXXXXXXXXXXIXGRXXXXXXXXXXXXXXXXXXXXC

SEQ ID NO: 2 - protein, 17 aa, Clostridium botulinum
CHKAIDGRSLYNKTLDC

SEQ ID NO: 3 - protein, 17 aa, Artificial Sequence
  C1 Activation Loop Variant
CHKAIEGRSLYNKTLDC

SEQ ID NO: 4 - DNA, 3891 bp, Artificial Sequence
  BoNT/X with a C1 Activation Loop
atgaaactggaaatcaacaaattcaactacaacgatccgatcgatggcattaatgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcatgaactggttcatgttacccataatctgtatggtattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgatttccaaggtcagctgctggaaagcagctattttgaaaaaattgaaagcaatgccctgcgtgcctttatcaaaatttgtcataaagccattgatggtcgcagcctgtataacaaaaccctggattgtattgaggtggaaaacaaagacctgtttctgattagcaacaaagatagcctgaacgatattaacctgagcgaagaaaaaatcaaaccggaaaccaccgtgttcttcaaagataaactgcctccgcaggatattacgctgagcaattatgattttaccgaagccaatagcattccgagcattagccagcagaacattctggaacgtaatgaagaactgtatgaaccgattcgcaatagcctgtttgaaatcaaaaccatctatgtggataagctgaccacctttcattttctggaagcccagaatattgatgagagcattgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaaccggcaaaatcgacgtgattgataaaagcagcgataccctggcaattgttccgtatattggtccgctgctgaatattggtaatgatattcgtcatggcgattttgtgggtgcaattgaactggcaggcattaccgcactgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaattctgctgaacaaaagcgttgaacaggccatgaaaaacaccgagaaattcatgattaaactgagcaacagctacctgaccaaagaaatgattccgaaagttcaggacaacctgaaaaactttgatctggaaaccaaaaagaccctggacaagttcatcaaagagaaagaagatatcctgggcaccaatctgagcagcagcctgcgtcgtaaagttagcattcgtctgaataaaaacattgccttcgacatcaacgatatcccgtttagcgaatttgatgatctgatcaaccagtacaaaaacgagatcgaagattatgaagtgctgaatctgggtgcagaagatggcaaaatcaaagatctgagcggtacaaccagcgatatcaatattggttcagatatcgaactggccgatggtcgtgaaaataaagcgattaagattaaaggcagcgagaacagcaccatcaaaattgcaatgaacaaatatctgcgttttagcgcgaccgataactttagcattagcttttggatcaaacatccgaaaccgaccaatctgcttaataacggtattgaatataccctggtcgagaactttaatcagcgtggttggaaaattagcatccaggatagcaaactgatttggtatctgcgcgatcacaataacagcatcaaaatcgttacaccggattatattgcgtttaatggctggaacctgattaccattacaaacaatcgtagcaaaggcagcatcgtgtatgttaacggtagcaaaattgaagagaaggacattagcagcatttggaataccgaagtggatgatccgattatcttccgcctgaaaaataaccgtgatacccaggcatttaccctgctggatcagtttagcatttatcgcaaagaactgaaccagaacgaagtggtgaaactgtataactactacttcaacagcaactacattcgcgatatttggggtaatccgctgcagtacaacaaaaaatactatctgcagacccaggacaaacctggtaaaggtctgatccgcgaatattggagcagctttggttatgattatgtgattctgagcgatagcaagaccattacctttccgaataatatccgttatggtgccctgtataatggtagcaaagtgctgatcaagaacagcaaaaaactggatggtctggtgcgcaataaagatttcattcagctggaaatcgatggctataatatgggtattagcgcagatcgctttaacgaggataccaactatattggcaccacctatggtacaacccatgatctgaccaccgattttgaaattattcagcgccaagagaaataccgcaattattgtcagctgaaaaccccgtataacatctttcataaaagcggtctgatgagcaccgaaaccagcaaaccgaccttccatgattatcgcgattgggtttatagcagcgcatggtattttcagaactatgaaaatctgaacctgcgcaaacataccaaaaccaactggtattttatcccgaaagatgaaggttgggatgaagatctggaagtgctgtttcagggtccgcatcatcaccaccatcaccatcatcatcactga

SEQ ID NO: 5 - protein, 1296 aa, Artificial Sequence
  BoNT/X with a C1 Activation Loop
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDEDLEVLFQGPHHHHHHHHHH

SEQ ID NO: 6 - DNA, 3993 bp, Artificial Sequence
  BoNT/XA [LHNX-HCA] with a C1 Activation Loop
atgaaactggaaatcaacaaattcaactacaacgatccgatcgatggcattaatgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcatgaactggttcatgttacccataatctgtatggtattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgattttcagggccagctgctggaaagcagctattttgaaaaaattgaatccaatgcgctgcgtgcctttatcaaaatttgtcataaagccattgatggtcgcagcctgtataacaaaaccctggattgtattgaagtggaaaacaaagacctgttcctgattagcaataaagatagcctgaacgatatcaacctgagcgaagaaaaaatcaaaccggaaaccaccgtgttcttcaaagataaactgcctccgcaggatattaccctgagcaattatgattttaccgaagccaatagcattccgagcattagccagcagaacattctggaacgtaatgaagaactgtatgaaccgattcgcaatagcctgtttgaaatcaaaaccatctatgtggataagctgaccacctttcattttctggaagcccagaatattgatgagagcattgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaaccggcaaaatcgacgtgattgataaaagcagcgataccctggccattgttccgtatattggtccgctgctgaatattggtaatgatattcgtcatggcgattttgtgggtgcaattgaactggcaggcattaccgcactgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaattctgctgaacaaaagcgttgaacaggccatgaaaaacaccgagaaattcatgattaaactgagcaacagctacctgaccaaagaaatgattccgaaagttcaggacaacctgaaaaactttgatctggaaaccaaaaagaccctggacaagttcatcaaagagaaagaagatatcctgggcaccaatctgagcagcagcctgcgtcgtaaagttagcattcgtctgaataaaaacattgccttcgacatcaacgatatcccgtttagcgaatttgatgatctgatcaaccagtacaaaaacgagatcgaagattatgaagtgctgaatctgggtgcagaagatggcaaaatcaaagatctgagcggtacaaccagcgatattaacattggtagcgatatcgaaatcatcaacaccagcattctgaatctgcgctatgaaagcaatcatctgattgatctgagccgttatgcgtccaaaatcaatattggcagcaaagtgaatttcgacccgatcgataaaaatcagatccagctgtttaatctggaaagctccaaaattgaggtgattctgaaaaacgcgattgtgtacaatagcatgtatgagaatttctcaaccagcttctggattcgcattccgaaatactttaacagcatcagcctgaacaacgagtataccattatcaactgcatggaaaacaatagcggttggaaagtgagcctgaattatggtgaaattatctggaccctgcaggatacccaagaaatcaaacagcgtgttgtgttcaaatacagccagatgattaacatcagcgattacattaaccgctggatctttgttaccattaccaacaatcgcctgaataacagcaagatctatattaacggtcgtctgattgaccagaaaccgattagtaatctgggtaatattcatgccagcaacaacatcatgttcaaactggatggttgtcgtgatacccatcgttatatttggatcaagtattttaacctgtttgataaagaactgaacgaaaaagaaattaaggatctgtatgataaccagtccaatagcggcatcctgaaggatttttggggtgattatctgcagtatgacaaaccgtattatatgctgaacctgtacgatccgaacaaatatgtggatgtgaataatgtgggtatccgtggctatatgtatctgaaaggtccgcgtggtagcgttatgaccaccaacatttatctgaatagcagcctgtatcgtggcaccaaattcatcatcaaaaaatacgccagcggcaacaaagataatattgtgcgtaataatgaccgcgtgtatatcaatgtggtggtgaagaataaagaatatcgtctggcaaccaatgcaagccaggcaggcgttgaaaaaattctgagcgcactggaaatcccggatgtgggtaatctgagccaggttgttgttatgaaaagcaaaaatgatcagggcatcaccaacaagtgcaaaatgaatctgcaggacaataacggcaacgacattggttttattggctttcaccagtttaacaacattgccaaactggttgcgagcaattggtataatcgtcagattgaacgtagcagtcgtaccctgggttgtagctgggaatttattccggttgatgatggttggggtgaacgtccgctgcatcatcaccaccatcaccatcaccaccattaa

SEQ ID NO: 7 - protein, 1330 aa, Artificial Sequence
  BoNT/XA [LHNX-HCA] with a C1 Activation Loop
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIEIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLHHHHHHHHHH

SEQ ID NO: 8 - DNA, 4017 bp, Artificial Sequence
  BoNT/XB [LHNX-HCB] with a C1 Activation Loop
atgaaactggaaatcaacaaattcaactacaacgatccgatcgatggcattaatgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcatgaactggttcatgttacccataatctgtatggtattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgattttcagggccagctgctggaaagcagctattttgaaaaaattgaatccaatgcgctgcgtgcctttatcaaaatttgtcataaagccattgatggtcgcagcctgtataacaaaaccctggattgtattgaagtggaaaacaaagacctgttcctgattagcaataaagatagcctgaacgatatcaacctgagcgaagaaaaaatcaaaccggaaaccaccgtgttcttcaaagataaactgcctccgcaggatattaccctgagcaattatgattttaccgaagccaatagcattccgagcattagccagcagaacattctggaacgtaatgaagaactgtatgaaccgattcgcaatagcctgtttgaaatcaaaaccatctatgtggataagctgaccacctttcattttctggaagcccagaatattgatgagagcattgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaaccggcaaaatcgacgtgattgataaaagcagcgataccctggccattgttccgtatattggtccgctgctgaatattggtaatgatattcgtcatggcgattttgtgggtgcaattgaactggcaggcattaccgcactgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaattctgctgaacaaaagcgttgaacaggccatgaaaaacaccgagaaattcatgattaaactgagcaacagctacctgaccaaagaaatgattccgaaagttcaggacaacctgaaaaactttgatctggaaaccaaaaagaccctggacaagttcatcaaagagaaagaagatatcctgggcaccaatctgagcagcagcctgcgtcgtaaagttagcattcgtctgaataaaaacattgccttcgacatcaacgatatcccgtttagcgaatttgatgatctgatcaaccagtacaaaaacgagatcgaagattatgaagtgctgaatctgggtgcagaagatggcaaaatcaaagatctgagcggtacaaccagcgatattaacattggtagcgatatcgaaatcctgaacaacattattctgaacctgcgctataaagataacaacctgattgatctgagtggctatggtgcaaaagttgaagtttatgatggtgtggaactgaacgacaaaaaccagttcaaactgaccagcagcgcaaattcaaaaattcgcgttacccagaaccagaacatcatttttaacagcgtgtttctggatttcagcgtgagcttttggattcgtattccgaaatataagaacgacggcatccagaactatatccacaatgaatataccatcatcaactgcatgaagaataacagcggttggaaaattagcatccgtggcaatcgtattatttggaccctgatcgatattaatggcaaaaccaagagcgtgtttttcgagtataacatccgtgaagatatcagcgaatacatcaaccgttggttttttgtgaccattaccaacaatctgaacaacgccaaaatctacattaacggcaaactggaaagcaacaccgatatcaaagatattcgtgaagtgattgccaacggcgagattatctttaaactggatggtgatattgatcgcacccagtttatttggatgaaatacttcagcatcttcaacaccgaactgagccagagcaatattgaagaacgctataaaatccagagctacagcgagtatctgaaagacttttggggtaatccgctgatgtacaacaaagaatactacatgtttaatgccggtaacaaaaacagctatatcaaactgaaaaaggatagtccggtgggtgaaattctgacccgtagcaaatataaccagaatagcaagtatatcaactatcgcgatctgtacatcggcgagaaatttatcattcgtcgtaaaagcaactcccagagcattaacgatgatattgtgcgcaaagaggattacatctacctggattttttcaacctgaatcaagagtggcgtgtgtacacctataagtacttcaaaaaagaagaaatgaaactgtttctggcaccgatctatgatagcgacgaattttacaataccattcagattaaagaatatgatgaacagccgacctatagctgtcagctgctgtttaaaaaggatgaagaaagcacggatgaaattggcctgattggtatccatcgtttttatgaaagcggcatcgtgttcgaagagtacaaagattatttctgcatcagcaaatggtatcttaaagaggtgaaacgcaaaccgtataatctgaaactgggttgcaattggcagttcatcccgaaagatgaaggttggaccgaacatcatcaccaccatcaccatcatcatcactga

SEQ ID NO: 9 - protein, 1338 aa, Artificial Sequence
  BoNT/XB [LHNX-HCB] with a C1 Activation Loop
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 10 - DNA, 3762 bp, Artificial Sequence
  BoNT/E with a C1 Activation Loop
atgccgaaaatcaactctttcaactacaacgacccggttaacgaccgtaccatcctgtatatcaaaccgggtggttgccaggagttctacaaatctttcaacatcatgaaaaacatctggatcatcccggaacgtaacgttatcggtaccaccccgcaggacttccacccgccgacctctctgaaaaacggtgactcttcttactacgacccgaactacctccagtctgacgaagaaaaagaccgtttcctgaaaatcgttaccaaaatcttcaaccgtatcaacaacaacctgtctggtggtatcctgctggaagaactgtctaaagctaacccgtacctgggtaacgacaacaccccggacaaccagttccacatcggtgacgcttctgctgttgaaatcaaattctctaacggttctcaggacatcctgctgccgaacgttatcatcatgggtgctgaaccggacctgttcgaaaccaactcttctaacatctctctgcgtaacaactacatgccgtctaaccacggtttcggttctatcgctatcgttaccttctctccggaatactctttccgtttcaacgacaacagcatgaacgagttcatccaggacccggctctgaccctgatgcaccaactgatctattctctgcacggtctgtacggtgctaaaggtatcaccaccaaatacaccatcacccagaaacagaacccgctgatcaccaacatccgtggtaccaacatcgaagagttcctgaccttcggtggtaccgacctgaacatcatcacctctgctcagtctaacgacatctacaccaacctgctggctgactacaaaaaaatcgcttctaaactgtctaaagttcaggtttctaacccgctgctgaacccgtacaaagacgttttcgaagctaaatacggtctggacaaagacgcttctggtatctactctgttaacatcaacaaattcaacgacatcttcaaaaaactgtactctttcaccgagttcgacctggcgaccaaattccaggttaaatgccgtcagacctacatcggtcagtacaaatacttcaaactgtctaacctgctgaacgactctatctacaacatctctgaaggttacaacatcaacaacctgaaagttaacttccgtggtcagaacgctaacctgaacccgcgtatcatcaccccgatcaccggtcgtggtctggttaaaaaaatcatccgtttctgccacaaagcgattgatggccgctctctctataacaaaacgctggattgcatcgaaatcaacaacggtgaactgttcttcgttgcttctgaaaactcttacaacgacgacaacatcaacaccccgaaagaaatcgacgacaccgttacctctaacaacaactacgaaaacgacctggaccaggttatcctgaacttcaactctgaatctgctccgggtctgtctgacgaaaaactgaacctgaccatccagaacgacgcttacatcccgaaatacgactctaacggtacctctgacatcgaacagcacgacgttaacgaactgaacgttttcttctacctggacgctcagaaagttccggaaggtgaaaacaacgttaacctgacctcttctatcgacaccgctctgctggaacagccgaaaatctacaccttcttctcttctgagttcatcaacaacgttaacaaaccggttcaggctgctctgttcgtttcttggattcagcaggttctggttgacttcaccaccgaagctaaccagaaatctaccgttgacaaaatcgctgacatctctatcgttgttccgtacatcggtctggctctgaacatcggtaacgaagctcagaaaggtaacttcaaagacgctctggaactgctgggtgctggtatcctgctggagttcgaaccggaactgctgatcccgaccatcctggttttcaccatcaaatctttcctgggttcttctgacaacaaaaacaaagttatcaaagctatcaacaacgctctgaaagaacgtgacgaaaaatggaaagaagtttactctttcatcgtttctaactggatgaccaaaatcaacacccagttcaacaaacgtaaagaacagatgtaccaggctctccagaaccaggttaacgctatcaaaaccatcatcgaatctaaatacaactcttacaccctggaagaaaaaaacgaactgaccaacaaatacgacatcaaacagatcgaaaacgaactgaaccagaaagtttctatcgctatgaacaacatcgaccgtttcctgaccgaatcttctatctcttacctgatgaaactcatcaacgaagttaaaatcaacaaactgcgtgaatacgacgaaaacgttaaaacctacctgctgaactacatcatccagcacggttctatcctgggtgaatctcagcaggaactgaactctatggttaccgacaccctgaacaactctatcccgttcaaactgtcttcttacaccgacgacaaaatcctgatctcttacttcaacaaattctttaaacgcattaagagttcatcggttctgaatatgcggtacaaaaatgataaatatgtcgatacttctggatatgatagcaatatcaacattaacggcgacgtgtataaatatccgacaaataaaaaccagtttgggatatataacgacaagctgtcggaggtcaatatttctcaaaacgactatatcatttacgataataaatataaaaactttagcattagtttttgggttcgtatacctaattatgacaataaaattgtaaatgtgaataacgagtataccattataaactgtatgcgcgacaataacagtggttggaaggtatcgctgaaccataatgagattatctggaccctgcaggataatgcaggtataaaccagaaactggcttttaactatggaaacgcaaatgggatctcagattacattaataaatggatttttgttaccattacgaacgatcgcttaggcgactcaaaactttatattaatggcaatctgatagatcagaaatcaatcttaaatttgggcaatattcatgtctctgataacatcttgttcaagatcgttaattgcagttacactcgttatattggcattcgttactttaatatcttcgataaagaactggacgagacggaaatccagactctgtattcaaacgagcccaatactaatatattgaaagatttttggggtaactatcttttatatgataaagaatactatctcctgaatgtattgaagccaaacaatttcatagatagacgcaaggatagcacattaagtatcaacaatatcagatctactatactgttagcaaatcgcctctactccggtattaaagtgaagattcagcgggttaataactccagtaccaatgataatctggtccgtaagaacgatcaggtatacatcaatttcgtcgcgagcaaaactcatctcttcccgctttacgccgatacagctacgacaaacaaggaaaaaaccataaaaatttccagctccggaaacagattcaatcaagtagttgtaatgaactctgtgggtaataattgtacgatgaactttaagaataacaatgggaacaatattggacttttgggcttcaaagccgacacagtggtggcgtccacctggtattacacgcacatgcgggaccatacgaattcgaacggttgcttctggaactttatctcggaagaacacgggtggcaagaaaaa

SEQ ID NO: 11 - protein, 1254 aa, Artificial Sequence
  BoNT/E with a C1 Activation Loop
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHQLIYSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCHKAIDGRSLYNKTLDCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 12 - DNA, 3867 bp, Artificial Sequence
  BoNT/A1C1 with a C1 Activation Loop
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgccacaaagcgattgatggccgctctctctataacaaaacgctggattgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaataa

SEQ ID NO: 13 - protein, 1288 aa, Artificial Sequence
  BoNT/A1C1 with a C1 Activation Loop
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAIDGRSLYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 14 - DNA, 3878 bp, Artificial Sequence
  BoNT/C1(0) (Endonegative)
atgccgatcacgattaataatttcaactatagcgatccggtggacaataagaatattctgtatctggatactcatctgaatacgctggctaacgaaccggagaaagcgttccgcatcacaggcaacatctgggttattcccgatcgcttttcacgcaacagcaaccctaatctgaacaaacctcctcgtgtcaccagtcctaaatccggttattacgacccaaactatctgagtacggatagcgataaagatccctttctgaaagagatcattaagctgttcaaacgcattaactctcgcgaaattggggaagagctgatctatcggctttcgacagatatcccgttcccaggtaacaataataccccgattaatactttcgactttgatgttgatttcaattctgtggatgtgaaaacgcgtcaaggcaataattgggtgaaaactggtagcattaacccgagtgtaattatcacaggtccccgtgagaacatcatcgacccggaaacctctaccttcaagctgacgaacaacacgtttgctgcacaggaagggtttggtgccctgtcaatcatttccatctcaccgcgtttcatgttaacctactccaatgccacaaatgatgttggcgaaggacgttttagcaaatcagaattttgcatggacccaattctcattctgatgggcacgctgaacaatgcgatgcacaacttgtatggcattgctattccaaacgatcaaaccattagctccgttaccagtaatatcttctatagccagtataatgtcaaattggagtatgccgaaatttacgcctttggaggcccgaccattgacctgattccgaaatctgcacgcaaatacttcgaagaaaaggcgttagattactatcgcagcatcgcgaaacgcctgaactcgattaccacggccaatccgtcgtcgttcaacaaatacattggtgaatataaacagaaactgattcgcaaatatcggtttgtcgtagaaagctctggtgaagtgactgtaaaccgcaacaaatttgtcgaactctacaacgagttgacccaaatctttaccgagtttaactacgcaaagatctataacgtacagaaccgcaagatttatcttagcaatgtatacacaccggttactgcgaacatcttagacgacaatgtgtatgatattcagaatggctttaacatcccgaaatcaaatctgaacgttctgtttatgggccagaacctgagtcgtaatccagcactgcgtaaagtgaacccggaaaatatgctctacttgtttaccaaattttgccacaaagcgattgatggccgctctctctataacaaaacgctggattgtcgtgagttacttgtgaagaacactgatttaccgttcattggggatatctccgacgtgaaaaccgatatcttcctgcgcaaagacattaatgaagaaacggaagtcatctattaccccgacaatgtgagcgttgatcaggtcattttatcgaagaacacctccgaacatggtcagttggatttgctgtaccctagcattgactcggagagtgaaatccttccgggcgaaaatcaagtgttttacgacaaccgtacccaaaatgttgattatttgaattcttattactacctggaatctcagaaattgagcgacaatgtggaagatttcacgttcacacgctccattgaggaagcgctggataatagcgcgaaagtgtatacgtatttccctaccttggcgaataaagtaaatgctggtgtccagggaggcttatttctgatgtgggcgaatgatgtggtagaagattttacgaccaatattttgcgtaaggacaccttagataaaattagcgatgttagcgccatcatcccctatattggcccagcactgaatatctcgaactctgtgcgtcgcggaaacttcaccgaagcatttgcggtgaccggggttactattctgttggaagcctttccggagtttactattccggcgctgggtgcgtttgtgatttattcgaaagtacaagaacgcaatgaaattatcaaaaccatcgataattgcctggaacaacgcattaaacgctggaaggattcttatgaatggatgatgggcacctggttatcccgtattatcacacagtttaacaacatctcgtatcagatgtacgattcactgaactaccaagcaggggcgatcaaagccaagatcgacttagaatacaagaaatattcaggtagcgataaagagaatattaaaagccaggttgaaaacctgaagaactctctggatgtcaaaatttcagaggctatgaacaacattaacaaatttatccgcgaatgtagcgtcacgtatctgtttaaaaacatgctcccgaaagtgattgatgagctcaacgagtttgatcgcaacacaaaggccaaactgattaacctgattgatagtcacaatattattttagtcggtgaagttgacaagctgaaggctaaggtcaataacagctttcagaacactattccgtttaatattttctcctatacgaacaatagtctgctgaaagacattatcaacgaatacttcaacaatattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaataatg

SEQ ID NO: 15 - protein, 1291 aa, Artificial Sequence
  BoNT/C1(0) (Endonegative)
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMGTLNNAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 16 - DNA, 3876 bp, Clostridium botulinum
atgccgatcacgattaataatttcaactatagcgatccggtggacaataagaatattctgtatctggatactcatctgaatacgctggctaacgaaccggagaaagcgttccgcatcacaggcaacatctgggttattcccgatcgcttttcacgcaacagcaaccctaatctgaacaaacctcctcgtgtcaccagtcctaaatccggttattacgacccaaactatctgagtacggatagcgataaagatccctttctgaaagagatcattaagctgttcaaacgcattaactctcgcgaaattggggaagagctgatctatcggctttcgacagatatcccgttcccaggtaacaataataccccgattaatactttcgactttgatgttgatttcaattctgtggatgtgaaaacgcgtcaaggcaataattgggtgaaaactggtagcattaacccgagtgtaattatcacaggtccccgtgagaacatcatcgacccggaaacctctaccttcaagctgacgaacaacacgtttgctgcacaggaagggtttggtgccctgtcaatcatttccatctcaccgcgtttcatgttaacctactccaatgccacaaatgatgttggcgaaggacgttttagcaaatcagaattttgcatggacccaattctcattctgatgcacgagctgaaccatgcgatgcacaacttgtatggcattgctattccaaacgatcaaaccattagctccgttaccagtaatatcttctatagccagtataatgtcaaattggagtatgccgaaatttacgcctttggaggcccgaccattgacctgattccgaaatctgcacgcaaatacttcgaagaaaaggcgttagattactatcgcagcatcgcgaaacgcctgaactcgattaccacggccaatccgtcgtcgttcaacaaatacattggtgaatataaacagaaactgattcgcaaatatcggtttgtcgtagaaagctctggtgaagtgactgtaaaccgcaacaaatttgtcgaactctacaacgagttgacccaaatctttaccgagtttaactacgcaaagatctataacgtacagaaccgcaagatttatcttagcaatgtatacacaccggttactgcgaacatcttagacgacaatgtgtatgatattcagaatggctttaacatcccgaaatcaaatctgaacgttctgtttatgggccagaacctgagtcgtaatccagcactgcgtaaagtgaacccggaaaatatgctctacttgtttaccaaattttgccacaaagcgattgatggccgctctctctataacaaaacgctggattgtcgtgagttacttgtgaagaacactgatttaccgttcattggggatatctccgacgtgaaaaccgatatcttcctgcgcaaagacattaatgaagaaacggaagtcatctattaccccgacaatgtgagcgttgatcaggtcattttatcgaagaacacctccgaacatggtcagttggatttgctgtaccctagcattgactcggagagtgaaatccttccgggcgaaaatcaagtgttttacgacaaccgtacccaaaatgttgattatttgaattcttattactacctggaatctcagaaattgagcgacaatgtggaagatttcacgttcacacgctccattgaggaagcgctggataatagcgcgaaagtgtatacgtatttccctaccttggcgaataaagtaaatgctggtgtccagggaggcttatttctgatgtgggcgaatgatgtggtagaagattttacgaccaatattttgcgtaaggacaccttagataaaattagcgatgttagcgccatcatcccctatattggcccagcactgaatatctcgaactctgtgcgtcgcggaaacttcaccgaagcatttgcggtgaccggggttactattctgttggaagcctttccggagtttactattccggcgctgggtgcgtttgtgatttattcgaaagtacaagaacgcaatgaaattatcaaaaccatcgataattgcctggaacaacgcattaaacgctggaaggattcttatgaatggatgatgggcacctggttatcccgtattatcacacagtttaacaacatctcgtatcagatgtacgattcactgaactaccaagcaggggcgatcaaagccaagatcgacttagaatacaagaaatattcaggtagcgataaagagaatattaaaagccaggttgaaaacctgaagaactctctggatgtcaaaatttcagaggctatgaacaacattaacaaatttatccgcgaatgtagcgtcacgtatctgtttaaaaacatgctcccgaaagtgattgatgagctcaacgagtttgatcgcaacacaaaggccaaactgattaacctgattgatagtcacaatattattttagtcggtgaagttgacaagctgaaggctaaggtcaataacagctttcagaacactattccgtttaatattttctcctatacgaacaatagtctgctgaaagacattatcaacgaatacttcaacaatattaatgacagcaaaattctgagcctgcagaatcgtaagaatacgctggtagataccagtggatataatgcggaagtctcagaagagggtgatgtacagctgaacccgatctttccgttcgactttaaactggggtctagtggtgaagatcgcggtaaagtgatcgttacccaaaacgagaacattgtgtataacagcatgtacgagagtttctcaatttctttctggattcgcatcaataaatgggtttctaatttgcctggctataccatcattgatagcgtcaaaaacaactcgggctggtcgattggcattattagcaactttctggtgtttaccctgaaacagaatgaggattcggaacagagcattaacttctcctacgacatcagcaacaatgcaccagggtataacaaatggttcttcgtaacggtgacgaacaatatgatgggcaatatgaaaatctacattaacgggaaacttatcgacaccattaaagtgaaagagcttactgggatcaattttagtaaaaccattacctttgagatcaacaaaattccggacacgggtctgattacctccgattcggataatatcaatatgtggattcgcgacttttatatcttcgccaaagaacttgatggcaaagatatcaacattttgtttaattccctgcagtataccaatgtcgttaaggactattggggcaatgatctccgctacaataaagaatactacatggttaacatcgactatctcaatcgctacatgtatgctaactcgcgtcaaattgtgtttaacacacgtcgtaacaacaacgattttaacgaaggttataaaatcattatcaaacggatccgcggcaatacgaacgatactcgtgttcgtggcggtgacattctgtatttcgacatgacgattaataataaagcgtacaatctgttcatgaagaacgaaaccatgtacgccgataaccattccactgaagatatctacgcaatcggacttcgcgaacagaccaaagacattaacgacaacatcatctttcagattcaaccgatgaataatacctactactatgcctcccagatcttcaaaagtaatttcaacggcgaaaacatttcaggcatttgctcaatcggcacttatcggttccggttaggtggtgattggtatcgtcacaactaccttgttcccacagtgaaacaaggcaactatgcatcgctcttagaaagcacatctacgcattggggttttgtgccagtcagtgaataa

SEQ ID NO: 17 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 18 - protein, 4 aa, Artificial Sequence
  Enterokinase and Factor Xa Cleavage Site
IDGR

SEQ ID NO: 19 - protein, 4 aa, Artificial Sequence
  Enterokinase and Factor Xa Cleavage Site Variant
IEGR

SEQ ID NO: 20 - protein, 45 aa, Clostridium botulinum
CPRNGLLYNAIYRNSKNYLNNIDLEDKKTTSKTNVSYPCSLLNGC

SEQ ID NO: 21 - protein, 25 aa, Clostridium botulinum
CVRGIITSKTKSLDKGYNKALNDLC

SEQ ID NO: 22 - protein, 10 aa, Clostridium botulinum
CKSVRAPGIC

SEQ ID NO: 23 - protein, 14 aa, Clostridium botulinum
CLRLTKNSRDDSTC

SEQ ID NO: 24 - protein, 15 aa, Clostridium botulinum
CKNIVSVKGIRKSIC

SEQ ID NO: 25 - protein, 17 aa, Clostridium botulinum
CKSVIPRKGTKAPPRLC

SEQ ID NO: 26 - protein, 17 aa, Clostridium botulinum
CKSIIPRKGTKQSPSLC

SEQ ID NO: 27 - protein, 17 aa, Clostridium botulinum
CKSIIPRKGTKAPPRLC

SEQ ID NO: 28 - protein, 15 aa, Clostridium botulinum
CLNSSFKKNTKKPLC

SEQ ID NO: 29 - protein, 15 aa, Clostridium botulinum
CKSIVSKKGTKNSLC

SEQ ID NO: 30 - protein, 29 aa, Clostridium tetani
CKKIIPPTNIRENLYNRTASLTDLGGELC

SEQ ID NO: 31 - protein, 15 aa, Clostridium botulinum
CKPVMYKNTGKSEQC

SEQ ID NO: 32 - DNA, 3972 bp, Artificial Sequence
  Wild-Type BoNT/X-10HT
atgaaactggaaatcaacaaattcaactacaacgatccgatcgatggcattaatgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcatgaactggttcatgttacccataatctgtatggtattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgatttccaaggtcagctgctggaaagcagctattttgaaaaaattgaaagcaatgccctgcgtgccttcattaaaatctgtccgcgtaatggtctgctgtataatgccatttatcgcaacagcaaaaactacctgaacaacattgatctggaagataaaaagaccacgagcaaaaccaatgttagctatccgtgtagcctgctgaatggttgtattgaagttgaaaacaaagacctgttcctgattagcaacaaagatagcctgaacgatattaacctgagcgaagaaaaaatcaaaccggaaaccaccgtgttcttcaaagataaactgcctccgcaggatattacgctgagcaattatgattttaccgaagccaatagcattccgagcattagccagcagaacattctggaacgtaatgaagaactgtatgaaccgattcgcaatagcctgtttgaaatcaaaaccatctatgtggataagctgaccacctttcattttctggaagcccagaatattgatgagagcattgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaaccggcaaaatcgacgtgattgataaaagcagcgataccctggcaattgttccgtatattggtccgctgctgaatattggtaatgatattcgtcatggcgattttgtgggtgcaattgaactggcaggcattaccgcactgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacatcgatgataaagccaaaatcaaaaacgccatcagcgaaaccgaaatcctgctgaacaaaagcgttgaacaggcaatgaaaaacaccgagaaattcatgatcaaactgagcaacagctatctgaccaaagaaatgattccgaaagtgcaggataacctgaaaaatttcgatctggaaaccaagaaaaccctggacaaatttatcaaagagaaagaggacattctgggcaccaatctgagcagcagcctgcgtcgtaaagttagcattcgtctgaataaaaacattgccttcgacatcaacgatatcccgtttagcgaatttgatgatctgatcaaccagtacaaaaacgagatcgaagattatgaagtgctgaatctgggtgcagaagatgggaaaatcaaagatctgagcggtacaaccagcgatatcaatattggttcagatatcgaactggccgatggtcgtgaaaataaagccattaagattaaaggcagcgagaacagcaccatcaaaattgcaatgaacaaatatctgcgttttagcgcgaccgataactttagcattagcttttggatcaaacatccgaaaccgaccaatctgcttaataacggtattgaatataccctggtcgagaactttaatcagcgtggttggaaaattagcatccaggatagcaaactgatttggtatctgcgcgatcacaataacagcatcaaaatcgttacaccggattatattgcgtttaatggctggaacctgattaccattacaaacaatcgtagcaaaggcagcattgtgtatgtgaacggtagcaaaattgaagagaaggatattagcagcatctggaataccgaagtggatgatccgattatctttcgcctgaaaaacaatcgcgatacccaggcgtttaccctgctggatcagtttagcatttatcggaaagaactgaaccagaacgaagtggtgaaactgtataactactacttcaacagcaactacattcgcgatatttggggtaatccgctgcagtacaacaaaaaatactatctgcagacccaggacaaacctggtaaaggtctgatccgcgaatattggagcagctttggttatgattatgtgattctgagcgatagcaagacgattacctttccgaataatatccgttatggtgccctgtataacggcagcaaagttctgatcaaaaatagcaaaaaactggatggtctggtgcgcaataaagatttcattcagctggaaatcgatggctataatatgggtattagcgcagatcgctttaacgaggataccaactatattggcaccacctatggtacaacccatgatctgaccaccgattttgaaattattcagcgccaagagaaataccgcaattattgtcagctgaaaaccccgtataacatctttcataaaagcggtctgatgagcaccgaaaccagcaaaccgacctttcatgattatcgtgactgggtttatagcagcgcatggtattttcagaactatgaaaatctgaacctgcgcaaacataccaaaaccaactggtattttatcccgaaagatgaaggttgggatgaagatcttgaagttctgtttcagggtccgcatcatcaccaccatcaccatcatcatcac

SEQ ID NO: 33 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 34 - protein, 1324 aa, Artificial Sequence
  Wild-Type BoNT/X-10HT
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDEDLEVLFQGPHHHHHHHHHH

SEQ ID NO: 35 - protein, 1296 aa, Clostridium botulinum [sequence omitted]

SEQ ID NO: 36 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 37 - protein, 1291 aa, Clostridium botulinum; identical to SEQ ID NO: 17

SEQ ID NO: 38 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 39 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 40 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 41 - protein, 1297 aa, Clostridium botulinum
  SITE (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 42 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 43 - DNA, 2634 bp, Artificial Sequence
  LHN/A1 with an EK Cleavage Site
atggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctgacgatgacgataaaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaagcacaccatcatcaccaccatcaccatcaccat

SEQ ID NO: 44 - protein, 878 aa, Artificial Sequence
  LHN/A1 with an EK Cleavage Site
MEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEAHHHHHHHHHH

SEQ ID NO: 45 - DNA, 3996 bp, Clostridium botulinum
atgaaactggaaatcaacaaattcaactacaacgatccgatcgatggcattaatgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcatgaactggttcatgttacccataatctgtatggtattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgatttccaaggtcagctgctggaaagcagctattttgaaaaaattgaaagcaatgccctgcgtgccttcattaaaatctgtccgcgtaatggtctgctgtataatgccatttatcgcaacagcaaaaatctggaagttctgtttcagggtccgcatcatcaccaccatcaccatcatcatcacctggaagtgttatttcagggaccgtatctgaataacattgatctggaagataaaaagaccacgagcaaaaccaatgttagctatccgtgtagcctgctgaatggttgtattgaagttgaaaacaaagacctgttcctgattagcaacaaagatagcctgaacgatattaacctgagcgaagaaaaaatcaaaccggaaaccaccgtgttcttcaaagataaactgcctccgcaggatattacgctgagcaattatgattttaccgaagccaatagcattccgagcattagccagcagaacattctggaacgtaatgaagaactgtatgaaccgattcgcaatagcctgtttgaaatcaaaaccatctatgtggataagctgaccacctttcattttctggaagcccagaatattgatgagagcattgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaaccggcaaaatcgacgtgattgataaaagcagcgataccctggcaattgttccgtatattggtccgctgctgaatattggtaatgatattcgtcatggcgattttgtgggtgcaattgaactggcaggcattaccgcactgctggaatatgttccggaatttaccattccgattctggttggtctggaagtgattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaattctgctgaacaaaagcgttgaacaggccatgaaaaacaccgagaaattcatgattaaactgagcaacagctacctgaccaaagaaatgattccgaaagttcaggacaacctgaaaaactttgacctggaaaccaaaaaaaccctggacaagttcatcaaagagaaagaagatatcctgggcaccaatctgagcagcagcctgcgtcgtaaagttagcattcgtctgaataaaaacattgccttcgacatcaacgatatcccgtttagcgaatttgatgatctgatcaaccagtacaaaaacgagatcgaagattatgaagtgctgaatctgggtgcagaagatggcaaaatcaaagatctgagcggtacaaccagcgatatcaatattggttcagatatcgaactggccgatggtcgtgaaaataaagccattaagattaaaggcagcgagaacagcaccatcaaaattgcaatgaacaaatatctgcgttttagcgccaccgataactttagcattagcttttggatcaaacatccgaaaccgaccaatctgcttaataacggtattgaatataccctggtcgagaactttaatcagcgtggttggaaaattagcatccaggatagcaaactgatttggtatctgcgcgatcacaataacagcatcaaaatcgttacaccggattatattgcgtttaatggctggaacctgatcaccattacgaataatcgtagcaaaggcagcatcgtgtatgtgaatggtagcaaaattgaagagaaggacattagcagcatttggaataccgaagtggatgatccgattatcttccgcctgaaaaataaccgtgatacccaggcatttaccctgctggatcagtttagcatttatcggaaagaactgaaccagaacgaagtggtgaaactgtataactactacttcaacagcaactacattcgcgatatttggggtaatccgctgcagtacaacaaaaaatactatctgcagacccaggacaaacctggtaaaggtctgatccgcgaatattggagcagctttggttatgattatgtgattctgagcgatagcaagacgattacctttccgaacaatatccgttatggtgccctgtataacggtagcaaagttctgatcaagaacagcaagaaattagatggtctggtgcgcaataaagatttcattcagctggaaatcgatggctataatatgggtattagcgcagatcgctttaacgaggataccaactatattggcaccacctatggtacaacccatgatctgaccaccgattttgaaattattcagcgccaagagaaataccgcaattattgtcagctgaaaaccccgtataacatctttcataaaagcggtctgatgagcaccgaaaccagcaaaccgaccttccatgattatcgcgattgggtttatagcagcgcatggtattttcagaactatgaaaatctgaacctgcgcaaacataccaaaaccaactggtattttatcccgaaagatgaaggttgggacgaagat

SEQ ID NO: 46 - protein, 1332 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNLEVLFQGPHHHHHHHHHHLEVLFQGPYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 47 - protein, 247 aa, Artificial Sequence
  Trypsin
MHPLLILAFVGAAVAFPSDDDDKIVGGYTCAENSVPYQVSLNAGYHFCGGSLINDQWVVSAAHCYQYHIQVRLGEYNIDVLEGGEQFIDASKIIRHPKYSSWTLDNDILLIKLSTPAVINARVSTLALPSACASGSTECLISGWGNTLSSGVNYPDLLQCLEAPLLSHADCEASYPGEITNNMICAGFLEGGKDSCQGDSGGPVACNGQLQGIVSWGYGCAQKGKPGVYTKVCNYVDWIQETIAANS

SEQ ID NO: 48 - protein, 269 aa, Artificial Sequence
  Lys-C
GVSGSCNIDVVCPEGNGHRDVIRSVAAYSKQGTMWCTGSLVNNSANDKKMYFLTANHCGMTTAAIASSMVVYWNYQNSTCRAPGSSSSGANGDGSLAQSQTGAVVRATNAASDFTLLELNTAANPAYNLFWAGWDRRDQNFAGATAIHHPNVAEKRISHSTVATEISGYNGATGTSHLHVFWQASGGVTEPGSSGSPIYSPEKRVLGQLHGGPSSCSATGADRSDYYGRVFTSWTGGGTSATRLSDWLDAAGTGAQFIDGLDSTGTPPV

SEQ ID NO: 49 - protein, 235 aa, Artificial Sequence
  Enterokinase Light Chain
IVGGSDSREGAWPWVVALYFDDQQVCGASLVSRDWLVSAAHCVYGRNMEPSKWKAVLGLHMASNLTSPQIETRLIDQIVINRHYNKRRKNNDIAMMHLEMKVNYTDYIQPICLPEENQVFPPGRICSIAGWGALIYQGSTADVLQEADVPLLSNEKCQQQMPEYNITENMVCAGYDAGGVDSCQGDSGGPLMCQENNRWLLAGVTSFGYQCALPNRPGVYARVPRFTEWIQSFLH

SEQ ID NO: 50 - protein, 259 aa, Artificial Sequence
  Factor Xa Heavy Chain
IVGGRDCAEGECPWQALLVNEENEGFCGGTILNEFYVLTAAHCLHQAKRFTVRVGDRNTEQEEGNEMAHEVEMTVKHSRFVKETYDFDIAVLRLKTPIRFRRNVAPACLPEKDWAEATLMTQKTGIVSGFGRTHEKGRLSSTLKMLEVPYVDRSTCKLSSSFTITPNMFCAGYDTQPEDACQGDSGGPHVTRFKDTYFVTGIVSWGEGCARKGKFGVYTKVSNFLKWIDKIMKARAGAAGSRGHSEAPATWTVPPPLPL

SEQ ID NO: 51 - protein, 140 aa, Artificial Sequence
  Factor Xa Light Chain
ANSFLEEVKQGNLERECLEEACSLEEAREVFEDAEQTDEFWSKYKDGDQCEGHPCLNQGHCKDGIGDYTCTCAEGFEGKNCEFSTREICSLDNGGCDQFCREERSEVRCSCAHGYVLGDDSKSCVSTERFPCGKFTQGRS

SEQ ID NO: 52 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 53 - protein, 863 aa, Artificial Sequence
  LHN/A with a C1 Activation Loop
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAIDGRSLYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIK

SEQ ID NO: 54 - protein, 865 aa, Artificial Sequence
  LHN/B with a C1 Activation Loop
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCHKAIDGRSLYNKTLDCIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNS

SEQ ID NO: 55 - protein, 865 aa, Artificial Sequence
  LHN/D with a C1 Activation Loop
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCHKAIDGRSLYNKTLDCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFN

SEQ ID NO: 56 - protein, 847 aa, Artificial Sequence
  LHN/E with a C1 Activation Loop
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCHKAIDGRSLYNKTLDCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFK

SEQ ID NO: 57 - protein, 864 aa, Artificial Sequence
  LHN/F with a C1 Activation Loop
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCHKAIDGRSLYNKTLDCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYK

SEQ ID NO: 58 - protein, 865 aa, Artificial Sequence
  LHN/G with a C1 Activation Loop
  SITE (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCHKAIDGRSLYNKTLDCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYIS

SEQ ID NO: 59 - protein, 867 aa, Artificial Sequence
  LHN/TeNT with a C1 Activation Loop
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCHKAIDGRSLYNKTLDCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDV

SEQ ID NO: 60 - protein, 896 aa, Artificial Sequence
  LHN/X with a C1 Activation Loop
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIE

SEQ ID NO: 61 - protein, 14 aa, Artificial Sequence
  BoNT/DC Activation Loop
CLRLTRNSRDDSTC

SEQ ID NO: 62 - protein, 25 aa, Clostridium botulinum
CVRGIITSKTKSLDEGYNKALNELC

SEQ ID NO: 63 - protein, 25 aa, Clostridium botulinum
CVRGIITSKTKSLDEGYNKALNDLC

SEQ ID NO: 64 - protein, 25 aa, Clostridium botulinum
CVRGIIPFKTKSLDEGYNKALNYLC

SEQ ID NO: 65 - protein, 25 aa, Clostridium botulinum
CVRGIIPFKTKSLDEGYNKALNDLC

SEQ ID NO: 66 - protein, 11 aa, Clostridium botulinum
CSNSNTKNSLC

SEQ ID NO: 67 - protein, 15 aa, Clostridium botulinum
CTNIFSPKGIRKSIC

SEQ ID NO: 68 - protein, 15 aa, Clostridium botulinum
CKNIVFSKGITKSIC

SEQ ID NO: 69 - protein, 15 aa, Clostridium botulinum
CKNIVFSKGIRKSIC

SEQ ID NO: 70 - protein, 10 aa, Clostridium botulinum
CKSVKVPGIC

SEQ ID NO: 71 - protein, 10 aa, Clostridium botulinum
CKSVKAPGIC

SEQ ID NO: 72 - protein, 5 aa, Artificial Sequence
  Enterokinase Cleavage Site
DDDDK

SEQ ID NO: 73 - protein, 23 aa, Artificial Sequence
  Influenza Virus Haemagglutinin Translocation Domain
GLFGAIAGFIENGWEGMIDGWYG

SEQ ID NO: 74 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XDXXXLL

SEQ ID NO: 75 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLL

SEQ ID NO: 76 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXIL

SEQ ID NO: 77 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLM

SEQ ID NO: 78 - protein, 4 aa, Artificial Sequence
  Tyrosine-Based Motif
  SITE (2)..(3): Xaa can be any naturally occurring amino acid
  VARIANT (4)..(4): Xaa is a hydrophobic amino acid
YXXX

SEQ ID NO: 79 - protein, 7 aa, Artificial Sequence
  TEV Cleavage Site
ENLYFQG

SEQ ID NO: 80 - protein, 6 aa, Artificial Sequence
  Thrombin Cleavage Site
LVPRGS

SEQ ID NO: 81 - protein, 8 aa, Artificial Sequence
  PreScission Cleavage Site
LEVLFQGP

(19) United States
(12) Patent Application Publication
(10) Pub. No.: US 2022/0168242 A1
(43) Pub. Date: Jun. 2, 2022

(54) PHARMACEUTICALLY ACCEPTABLE SALTS OF MESDOPETAM AND USES THEREOF

(71) Applicants: Susanna Holm Waters, Gothenburg (SE); Joakim Mihkel Tedroff, Gothenburg (SE); Per Svenningsson, Stockholm (SE)

(72) Inventors: Susanna Holm Waters, Gothenburg (SE); Joakim Mihkel Tedroff, Gothenburg (SE); Per Svenningsson, Stockholm (SE)

(21) Appl. No.: 17/454,110

(22) Filed: Nov. 9, 2021

(30) Foreign Application Priority Data
Nov. 10, 2020 (EP) 20206645.2

(51) Int. Cl. A61K 31/137; A61K 45/06; A61P 25/16

(52) U.S. Cl. CPC A61K 31/137; A61P 25/16; A61K 45/06

(57) ABSTRACT

A method for the prevention or reduction of sensitization to a pharmaceutical drug for Parkinson's disease, the method comprising administering to a subject in need thereof a therapeutically effective amount of a compound of Formula I,

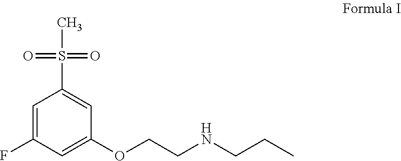 [SMILES: CCCNCCOc1cc(F)cc(S(C)(=O)=O)c1]

or a pharmaceutically acceptable salt thereof.

A method for the optimization of the dosage of a pharmaceutical drug for Parkinson's disease, the method comprising administering to a subject in need thereof a therapeutically effective amount of a compound of Formula I or a pharmaceutically acceptable salt thereof.

### BACKGROUND

Parkinson's disease is a chronic degenerative disease of the central nervous system that mainly affects the motor system. Common symptoms of the disease include tremor, rigidity, slowness of movement and difficulty with walking. These motor problems are commonly denominated “parkinsonism” or “parkinsonian syndrome”. In addition, non-motor related symptoms may occur such as depression, dysautonomia, sleep disorder, anxiety, fatigue and dementia.

Parkinson's disease, which may be abbreviated PD, is the second most common neurodegenerative disorder after Alzheimer's disease. Most people who are diagnosed with Parkinson's disease are over 60, and with the elderly as the fastest growing age group in many countries this poses a challenge to the health care systems.

The treatment of Parkinson's disease with L-DOPA, also called levodopa or L-3,4-dihydroxyphenylalanine, was developed in the 1960s and remains the gold standard for treatment. L-DOPA addresses the reduced dopamine production in the brain, which causes the motor symptoms of Parkinson's disease, by acting as a precursor that is able to cross the blood brain barrier after which it is converted into dopamine. Unfortunately, however, long-term use of L-DOPA is associated with dyskinesias which are rapid, uncontrolled involuntary movements different than the tremors associated with Parkinson's disease. Usually, the dyskinesias emerge after 4 to 10 years of medication against Parkinson's disease, and the severity can vary. The dyskinesias may be localized to a part of the body such as the face, arm and, leg, and are bothersome to the patients being affected. In clinical practice, the main strategy to reduce the L-DOPA induced dyskinesias (also called LIDs) is to reduce and adjust dopaminergic treatment to minimize fluctuations in plasma concentration of the drug since such fluctuations are believed to cause the dyskinesias. The lower dosage of L-DOPA reduces the dyskinesias but suffers from the drawback of non-optimal treatment of the motor symptoms associated with Parkinson's disease.

Due to the increased risk of developing adverse events such as dyskinesias at higher doses of L-DOPA, there is a large number of PD patients that are undertreated, i.e. many patients receive less than optimal doses of L-DOPA for the management of PD (see e.g. New Engl. J. Med. 351: 2498-2508).

Patients affected by LIDs have very few treatment alternatives available. In the USA, an extended-release formulation of amantadine was introduced against LIDs in 2017. However, amantadine is associated with severe side-effects such as hallucinations and falls.

WO 2012/143337 discloses phenoxy-ethyl-amine derivatives useful as modulators of cortical and basal ganglia dopaminergic and N-methyl-D-aspartate (NMDA) receptor mediated glutamatergic neurotransmission, and more specifically for the treatment of diseases that are responsive to modulation of dopaminergic and glutamatergic function in the central nervous system. The compound [2-(3-fluoro-5-methanesulfonylphenoxy)-ethyl](propyl)amine is disclosed in its non-salt form as well as in the form of a hydrochloric acid salt in Example 1. It is stated that said hydrochloric acid salt has a melting point of 191° C.

PCT/EP2020/064046 discloses pharmaceutically acceptable salts of the compound [2-(3-fluoro-5-methanesulfonylphenoxy)-ethyl](propyl)amine and their use in the treatment of e.g. Parkinson's disease, dyskinesias and L-DOPA induced dyskinesias. In particular, the tartaric acid salt of [2-(3-fluoro-5-methanesulfonylphenoxy)-ethyl](propyl)amine for use as a pharmaceutical drug was found to be non-hygroscopic allowing for storage without being changed by surrounding humidity.

J. Pharmacol. Exp. Ther. 374:113-125, July 2020 discloses the preclinical pharmacology of [2-(3-fluoro-5-methanesulfonylphenoxy)-ethyl](propyl)amine (also named IRL790) as a novel dopamine transmission modulator for the treatment of motor and psychiatric complications for Parkinson's disease. It was found that IRL790 dose-dependently reduced adverse involuntary movements (ATMs), and that this alleviation of involuntary movements was not achieved at the cost of any impairment of the motor effects of L-DOPA as such, which was captured as rotational response to L-DOPA.

WO 2020/110128 A1 discloses a combination of pridopidine and an additional therapeutic agent for treating drug-induced dyskinesia. It is described that the additional therapeutic agent may be IRL790.

Of course, it is a significant benefit that progress has been made in the treatment of dyskinesias so that these can be addressed when they start to develop. However, it would be even more desirable for patients suffering from PD if the treatment from the onset minimized the risk for development of dyskinesias such as LIDs thereby providing an overall improved treatment. This is particularly important for PD patients who are at greater risk for developing dyskinesias, such as patients diagnosed with PD at a younger age such as below the age of 60 years. Other independent risk factors for PD patients to develop LIDs are for instance cumulative L-DOPA exposure (e.g. according to Levodopa equivalent daily dose (LEDD)), female gender, severity of motor and functional impairment (e.g. as assessed by MDS-UPDRS Part III score), non-tremor dominant clinical phenotype, genetic risk score (e.g. polygenic risk score) and anxiety (see e.g. npj Parkinson's Disease. 33: 1-6 (2018), the entire contents of which are incorporated herein by reference). One possible advantageous way of minimizing the risk for development of LIDs may be by blocking the sensitization process being responsible for LIDs to occur in the first place. Such a therapy against PD and subsequently against occurring LIDs would thus be a disease modifying therapy that not only prevents LIDs from occurring, but also allows for a more optimal dose of L-DOPA. Moreover, it is desirable that the preparation used for the treatment exhibits good storage and/or handling properties.

### SUMMARY

It is an object of the present disclosure to provide a treatment preparation such as a pharmaceutical composition for treatment of Parkinson's disease while at the same minimizing the risk for development of dyskinesias such as LIDs. Further, it is an object of the present disclosure to provide a treatment preparation, such as the aforementioned pharmaceutical composition, which exhibits good storage and/or handling properties.

Thus, there is provided a pharmaceutical composition comprising a pharmaceutically acceptable salt of the compound [2-(3-fluoro-5-methanesulfonylphenoxy)ethyl](propyl)amine (also named IRL790 or mesdopetam), L-DOPA or a pharmaceutically acceptable salt thereof and optionally a pharmaceutically acceptable excipient, carrier and/or diluent.

In an aspect, there is provided a pharmaceutical composition comprising:
(i) a salt of Formula III:

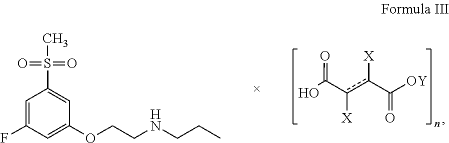

said salt being a combination of a compound of Formula I and an acid of Formula II:

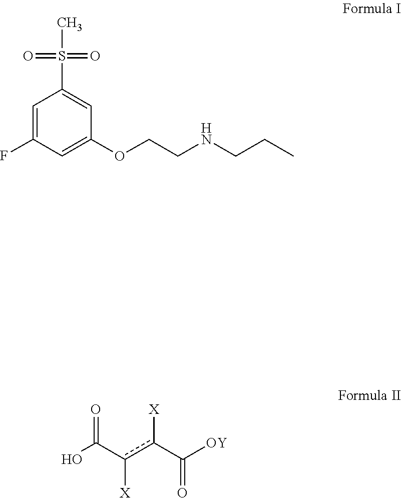

in a ratio of 1:n,
wherein
X is H or OH,
Y is H or a cation selected from the group consisting of Li, Na and K,
[symbol] a single bond or
a double bond, and
n is 0.5 or 1,
(ii) L-DOPA or a pharmaceutically acceptable salt thereof, and
(iii) a pharmaceutically acceptable excipient, carrier and/or diluent.

Further, there is provided a pharmaceutical composition as described herein for use in the treatment and/or prevention of Parkinson's disease.

There is also provided a use of a pharmaceutical composition as described herein for the manufacture of a medicament for use in the treatment and/or prevention of Parkinson's disease.

There is also provided a method for treatment and/or prevention of Parkinson's disease, said method comprising administering to a patient, such as a human or an animal, a therapeutically effective amount of the pharmaceutical composition as described herein.

In one aspect the present invention therefore also provides a compound of Formula I,

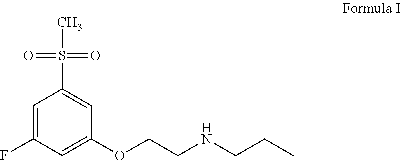 [SMILES: CCCNCCOc1cc(F)cc(S(C)(=O)=O)c1]

or a pharmaceutically acceptable salt thereof,
for use in the prevention or reduction of sensitization to a pharmaceutical drug for Parkinson's disease.

In another aspect the present invention also provides the use of a compound of Formula I,

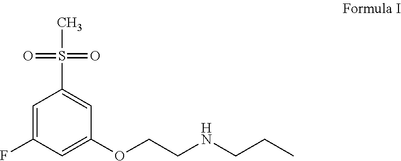 [SMILES: CCCNCCOc1cc(F)cc(S(C)(=O)=O)c1]

or a pharmaceutically acceptable salt thereof,
for the manufacture of a medicament for the prevention or reduction of sensitization to a pharmaceutical drug for Parkinson's disease.

In another aspect the present invention also provides a method for the prevention or reduction of sensitization to a pharmaceutical drug for Parkinson's disease, said method comprising administering to a subject in need thereof a therapeutically effective amount of a compound of Formula I

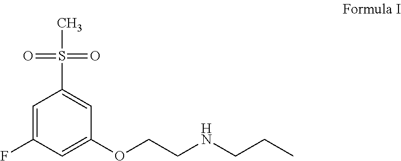 [SMILES: CCCNCCOc1cc(F)cc(S(C)(=O)=O)c1]

or a pharmaceutically acceptable salt thereof.

In another aspect the present invention also provides a compound of Formula I,

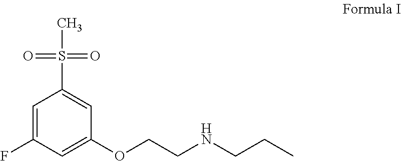 [SMILES: CCCNCCOc1cc(F)cc(S(C)(=O)=O)c1]

or a pharmaceutically acceptable salt thereof,
for use in optimization of the dosage of a pharmaceutical drug for Parkinson's disease.

In another aspect the present invention also provides a use of a compound of Formula I,

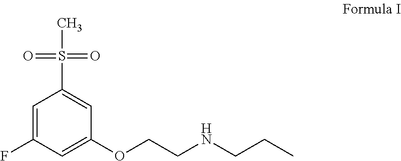 [SMILES: CCCNCCOc1cc(F)cc(S(C)(=O)=O)c1]

or a pharmaceutically acceptable salt thereof,
for the manufacture of a medicament for use in optimization of the dosage of a pharmaceutical drug for Parkinson's disease.

In a further aspect, the present invention also provides a method for the optimization of the dosage of a pharmaceutical drug for Parkinson's disease, said method comprising administering to a subject in need thereof a therapeutically effective amount of a compound of Formula I

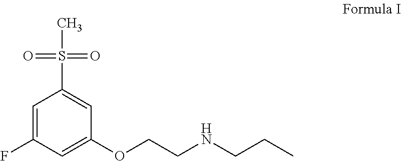 [SMILES: CCCNCCOc1cc(F)cc(S(C)(=O)=O)c1]

or a pharmaceutically acceptable salt thereof.

The present invention further provides numerous embodiments of these aspects, including those which are now outlined below.

In certain embodiments, the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease.

In certain embodiments, the pharmaceutical drug for Parkinson's disease comprises or consists of L-DOPA or a pharmaceutically acceptable salt thereof.

In certain embodiments, prevention or reduction of sensitization to a pharmaceutical drug for Parkinson's disease comprises or consists of prevention or reduction of L-DOPA sensitization.

In certain embodiments, prevention or reduction of sensitization to a pharmaceutical drug for Parkinson's disease comprises administering the compound of Formula I or a pharmaceutically acceptable salt thereof to a subject who has not experienced dyskinesias.

In certain embodiments, prevention or reduction of sensitization to a pharmaceutical drug for Parkinson's disease comprises administering the compound of Formula I or a pharmaceutically acceptable salt thereof to a subject who has not experienced L-DOPA induced dyskinesias.

In certain embodiments, prevention or reduction of sensitization to a pharmaceutical drug for Parkinson's disease comprises administering the compound of Formula I or pharmaceutically acceptable salt thereof to a subject who has been undergoing a course of treatment with a pharmaceutical drug for Parkinson's disease (e.g. L-DOPA or a pharmaceutically acceptable salt thereof) for at least 1 day prior to administration of the compound of Formula I or pharmaceutically acceptable salt thereof to the subject for the first time.

In certain embodiments, prevention or reduction of sensitization to a pharmaceutical drug for Parkinson's disease comprises administering the compound of Formula I or pharmaceutically acceptable salt thereof to a subject who has not previously been administered a pharmaceutical drug for Parkinson's disease. Optionally said pharmaceutical drug for Parkinson's disease is L-DOPA or a pharmaceutically acceptable salt thereof.

In certain embodiments, prevention or reduction of sensitization to a pharmaceutical drug for Parkinson's disease comprises administering the compound of Formula I or pharmaceutically acceptable salt thereof to the subject at least 1 day prior to administration of a pharmaceutical drug for Parkinson's disease (e.g. L DOPA or a pharmaceutically acceptable salt thereof) to the subject for the first time.

In certain embodiments, the compound of Formula I or pharmaceutically acceptable salt thereof is administered to a subject after the subject has been diagnosed with Parkinson's disease.

In certain embodiments, the compound of Formula I or pharmaceutically acceptable salt thereof is administered to a patient at risk for Parkinson's disease such as a patient with one or more following characteristics:

being below the age of about 60 years,
being a woman,
exhibiting severe motor and functional impairment,
with a genetic susceptibility for Parkinson's disease, suffering from anxiety.

In certain embodiments, the compound of Formula I or pharmaceutically acceptable salt thereof is administered in an amount corresponding to about 2.0 mg up to about 10.0 mg of the compound of Formula I, such as an amount corresponding to about 2.5 mg, about 5.0 mg or about 7.5 mg of the compound of Formula I.

In certain embodiments, the compound of Formula I or pharmaceutically acceptable salt thereof is administered in one or more doses in a first amount and then administered in one or more doses in a second amount, wherein said second amount is lower than said first amount. Optionally, the first amount corresponds to an amount of about 7.5 up to about 10.0 mg of the compound of Formula I and/or the second amount corresponds to an amount of about 2.5 to about 5.0 mg of the compound of Formula I.

In certain embodiments, the compound of Formula I or pharmaceutically acceptable salt thereof is administered twice per day.

In certain embodiments, the pharmaceutically acceptable salt of the compound of Formula I is a salt of Formula III:

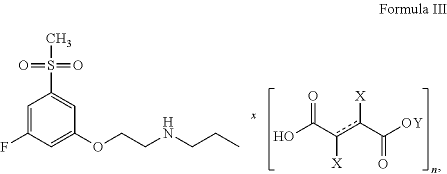

said salt being a combination of a compound of Formula I and an acid of Formula II:

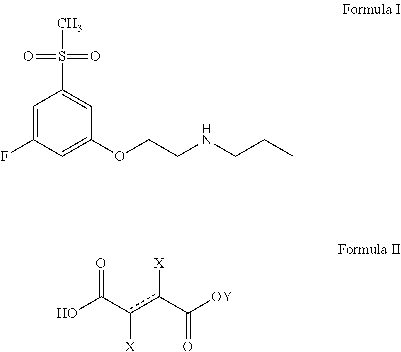

in a ratio of 1:n,
wherein

### X is H or OH,

Y is H or a cation selected from the group consisting of Li, Na and K, [symbol]
is a single bond or
a double bond, and
n is 0.5 or 1. Optionally, the following values apply for the salt of Formula III:

### X is OH,

### Y is H, and

[symbol] is a single bond,
thereby providing a salt of Formula IV being a combination of a compound of Formula I and tartaric acid:

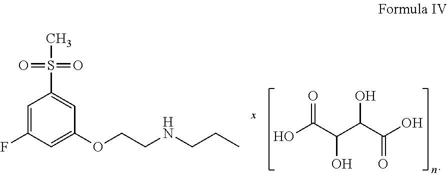

n is 0.5.

In certain embodiments, optimization of the dosage of a pharmaceutical drug for Parkinson's disease comprises or consists of optimization of the dosage of L-DOPA or a pharmaceutically acceptable salt thereof. Optionally, optimization of the dosage of a pharmaceutical drug for Parkinson's disease (e.g. L-DOPA or a pharmaceutically acceptable salt thereof) comprises administering the pharmaceutical drug for Parkinson's disease (e.g. L-DOPA or a pharmaceutically acceptable salt thereof) to a subject in an amount which is increased over time.

By “optimization of the dosage” is meant administering a dosage of a pharmaceutical drug for Parkinson's disease (e.g. L-DOPA or a pharmaceutically acceptable salt thereof) providing optimal treatment of the motor symptoms associated with Parkinson's disease. Such optimization might, for instance, be reflected in the administration of dosages which are higher than might conventionally be used due to the association of such dosages with dyskinesias (e.g. LIDs); or in maintaining a particular dosage level of a pharmaceutical drug for Parkinson's disease (e.g. L-DOPA or a pharmaceutically acceptable salt thereof) without reducing said dosage level; or in maintaining a particular dosage level of a pharmaceutical drug for Parkinson's disease (e.g. L-DOPA or a pharmaceutically acceptable salt thereof) for longer than is conventional; or in increasing the dosage amount of a pharmaceutical drug for Parkinson's disease (e.g. L-DOPA or a pharmaceutically acceptable salt thereof) which is administered over time.

### BRIEF DESCRIPTION OF THE DRAWINGS

FIG. 1 shows an XRP diffractogram of the salt of Formula IVa.

FIG. 2 shows an XRP diffractogram of the salt of Formula Va.

FIG. 3a shows the chemical structure for L-DOPA.

FIG. 3b shows the chemical structure for apomorphine.

FIG. 3c shows the chemical structure for pramipexole.

FIG. 3d shows the chemical structure for ropinirole.

FIG. 3e shows the chemical structure for rotigotine.

FIG. 3f shows the chemical structure for pridopidine.

FIG. 4a shows the rotational behaviour of rodents 0-2.5 hours post administration of test compound, namely saline, L-DOPA, the salt of Example 7, L-DOPA+ the salt of Example 7, amantadine, or L-DOPA+ amantadine, on Day 1.

FIG. 4b shows the rotational behaviour of rodents 0-2.5 hours post administration of test compound, namely saline, L-DOPA, the salt of Example 7, L-DOPA+ the salt of Example 7, amantadine, or L-DOPA+ amantadine, on Day 7.

FIG. 4c shows the rotational behaviour of rodents 0-2.5 hours post administration of test compound, namely saline, L-DOPA, the salt of Example 7, L-DOPA+ the salt of Example 7, amantadine, or L-DOPA+ amantadine, on Day 14.

FIG. 5 shows total number of contralateral turns in rats chronically treated with L-DOPA, L-DOPA+ the salt of Example 7, and L-DOPA+ amantadine for 2 weeks.

FIG. 6 shows striatal gene expression of the immediate early response gene (IEG) cfos (proto-oncogene, NM 022197) in 6-OHDA-lesioned rats following 14 days of treatment with vehicle, L-DOPA, IRL790 (the salt of Example 7, 3 mg/kg), amantadine or combinations of L-DOPA+ the salt of Example 7 (3 mg/kg) or L-DOPA+ amantadine. Data is expressed as relative gene expression and was statistically evaluated by two-way ANOVA (analysis of variance).

FIG. 7 shows striatal gene expression of the immediate early response gene (IEG) arc (activity-regulated cytoskeleton-related protein, U19866) in 6-OHDA-lesioned rats following 14 days of treatment with vehicle, L-DOPA, the salt of Example 7 (3 mg/kg), amantadine or combinations of L-DOPA+ the salt of Example 7 (3 mg/kg) or L-DOPA+ amantadine. Data is expressed as relative gene expression and was statistically evaluated by two-way ANOVA (analysis of variance). *denotes p<0.05, **denotes p<0.01, and *** denotes p<0.001.

### DESCRIPTION

The present disclosure provides a pharmaceutical composition comprising a pharmaceutically acceptable salt of the compound [2-(3-fluoro-5-methanesulfonylphenoxy)ethyl](propyl)amine (also named IRL790 or mesdopetam), L-DOPA or a pharmaceutically acceptable salt thereof and optionally a pharmaceutically acceptable excipient, carrier and/or diluent.

In an aspect, there is provided a pharmaceutical composition comprising: (i) a salt of Formula III:

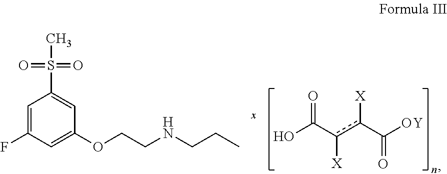

said salt being a combination of a compound of Formula I and an acid of Formula II:

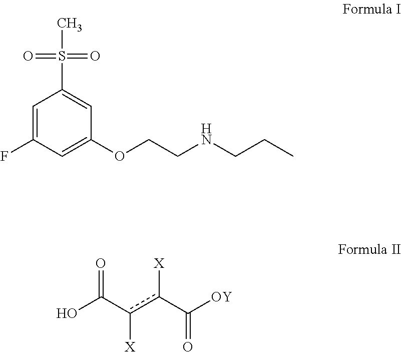

in a ratio of 1:n,
wherein
X is H or OH,
Y is H or a cation selected from the group consisting of Li, Na and K,
[symbol] is a single bond or
a double bond, and
n is 0.5 or 1,
(ii) L-DOPA or a pharmaceutically acceptable salt thereof, and
(iii) optionally a pharmaceutically acceptable excipient, carrier and/or diluent.

Further values for the salt of Formula III and components thereof, i.e. the compound of Formula I and the acid of Formula II, will now follow. As described herein, the salt of Formula III forms part of the pharmaceutical composition described herein.

It will be appreciated that the acid of Formula II described herein may be represented as.

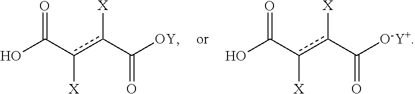

For example, when Y is H, i.e. hydrogen; the acid of Formula II may be represented as

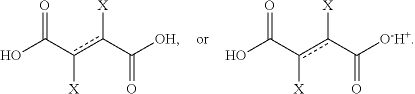

In a further example, when Y is Na, i.e. sodium, the acid of Formula II may be represented as

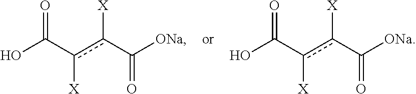

Further values of X, Y, [symbol] and n will now follow. It will be appreciated that such values may be used with any of the definitions, examples and/or claims described herein.

For instance, when

### X is OH,

### Y is H, and

[symbol] is a single bond,
the acid of Formula II is tartaric acid such as L-(+)-tartaric acid and/or D-(−)-tartaric acid. The tartaric acid may be combined with the compound of Formula I as described herein.

Thus, for the salt of Formula III when

### X is OH,

### Y is H, and

[symbol] is a single bond,
there is provided a salt of Formula IV being a combination of a compound of Formula I and tartaric acid:

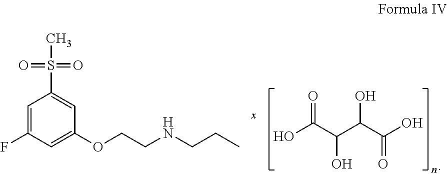

The tartaric acid described herein may be L-(+)-tartaric acid and/or D-(−)-tartaric acid. For instance, the tartaric acid may be L-(+)-tartaric acid. In a further example, the tartaric acid may be D-(−)-tartaric acid. In still a further example, the tartaric acid may be a mixture such as a racemic mixture of L-(+)-tartaric acid and D-(−)-tartaric acid.

The ratio of the compound of Formula I and the tartaric acid may be 1:n, i.e. the ratio of the compound of Formula I to the tartaric acid, wherein n is a number such as 0.5 or 1.

For instance, when the ratio of the compound of Formula I to the tartaric acid is 1:0.5 there is provided a salt of Formula IVa. In a further example, when the ratio of the compound of Formula I to the tartaric acid is 1:1 there is provided a salt of Formula IVb.

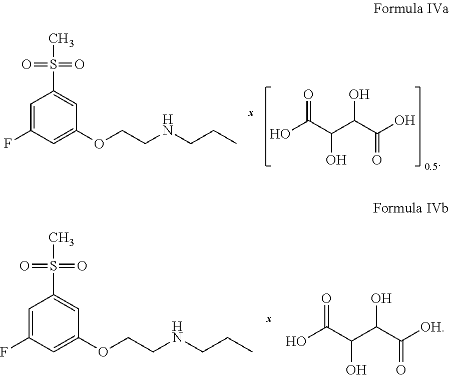

In a further example, when

### X is H,

### Y is H, and

[symbol] is a double bond,
the acid of Formula II is fumaric acid. The fumaric acid may be combined with the compound of Formula I as described herein.

Thus, for the salt of Formula III when

### X is H,

### Y is H, and

[symbol] is a double bond,
there is provided a salt of Formula V being a combination of a compound of Formula I and fumaric acid:

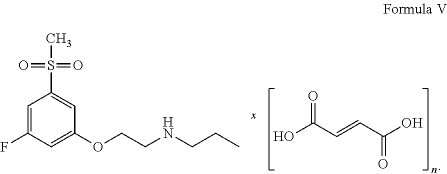

The ratio of the compound of Formula I and the fumaric acid may be 1:n, wherein n is a number such as 0.5 or 1. For example, n may be 0.5. In a further example, n may be 1.

For instance, when the ratio of the compound of Formula I to the fumaric acid is 1:0.5 there is provided a salt of Formula Va. In a further example, when the ratio of the compound of Formula I to the fumaric acid is 1:1 there is provided a salt of Formula Vb.

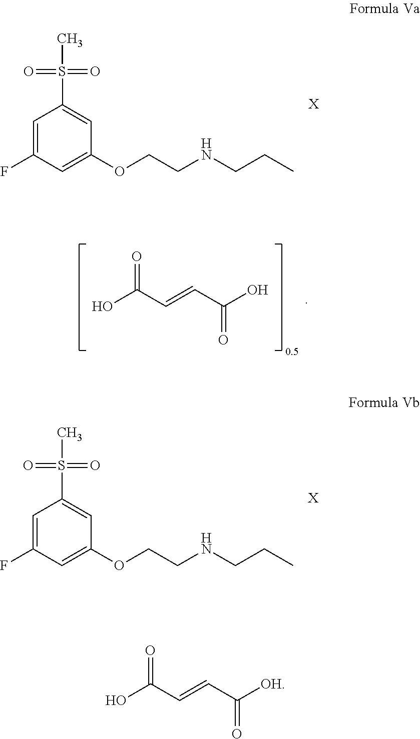

There is also provided a pharmaceutical composition as described herein comprising the salt of Formula III such as a salt of Formula IV or a salt of Formula V, wherein one or more of the hydrogen atoms of the compound of Formula I is/are replaced with deuterium. Additionally or alternatively, the salt of Formula III may be labelled with isotopes other than deuterium as described herein.

The salt of Formula III as described herein is pharmaceutically acceptable and has unexpectedly been found to exhibit properties of high crystallinity (i.e. being substantially crystalline), not being hygroscopic, exhibiting high melting point and/or satisfactory water solubility. Furthermore, the salt of Formula III may be isolated in good chemical yield with a high purity.

There is provided a salt of Formula III, as described herein, characterized by being crystalline. The crystallinity may be determined by XRPD or any other appropriate method known in the art. The high crystallinity of the salt of Formula III makes it well-defined with respect to, for instance, melting point and XRPD. This is a benefit in making tablets and is believed to enhance storage stability. In this document, high crystallinity intends a degree of crystallinity of about 80% or more such as about 85%, about 90%, about 95%, about 99% or about 100% as measured by XRPD or any other appropriate method of measurement known in the art.

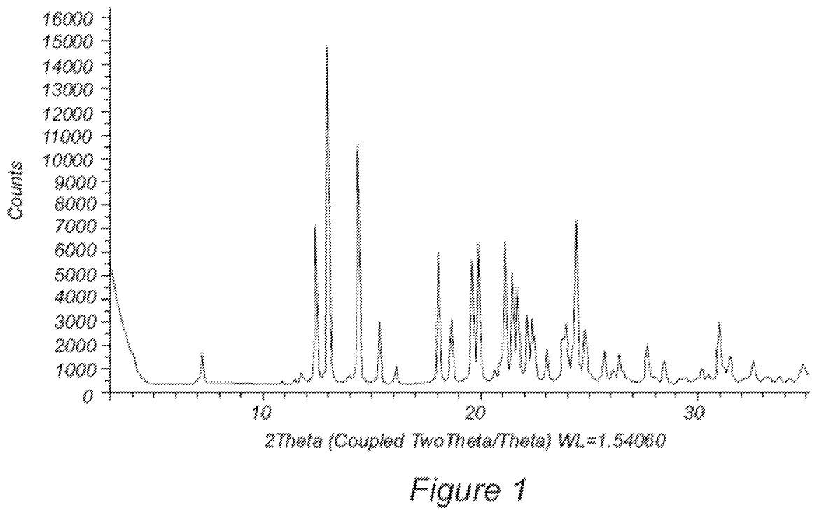

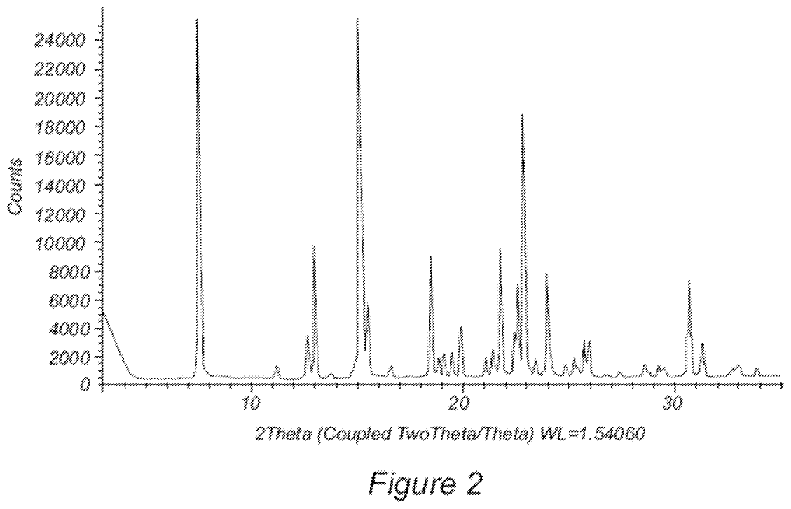

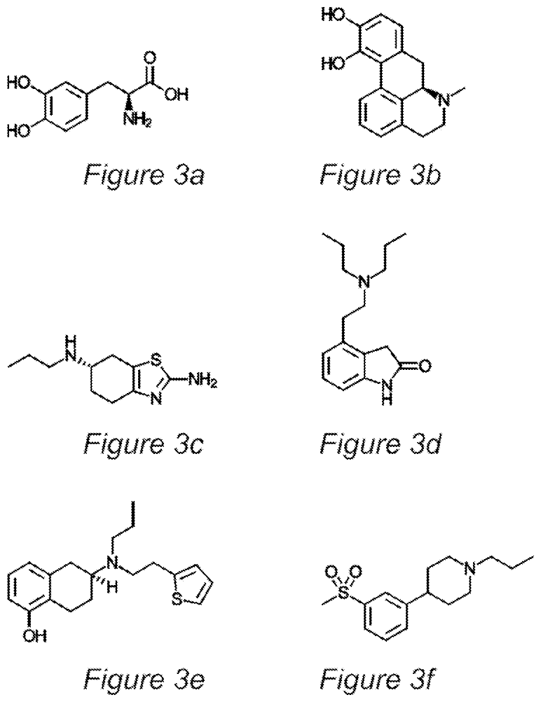

The salt of Formula III, as described herein, may be characterized by an XRP diffractogram as shown in FIG. 1 or in FIG. 2. The salt of Formula III such as the salt of Formula IVa may be characterized by an XRP diffractogram comprising a peak at about 13.02 2θ such as 13.0 2θ, and optionally at least one further peak selected from the following: about 12.43 such as about 12.4, about 14.40 such as about 14.4, about 21.10 such as about 21.1, about 24.36 such as about 24.4 2θ. The salt of Formula IVa may also be characterized by an XRP diffractogram comprising a peak at about 12.43, about 13.02, about 14.40, about 21.10, about 24.36 2θ, and optionally at least one further peak selected from the following: about 18.07, about 19.92 2θ. For instance, the XRP diffractogram may comprise peaks at about 12.4, about 13.0, about 14.4, about 21.1 and about 24.4 2θ. The salt of Formula IVa may also be characterized by an XRP diffractogram comprising a peak at about 12.43, about 13.02, about 14.40, about 18.07, about 19.92, about 21.10, about 24.36 2θ, and optionally at least one further peak selected from the following: about 19.62, about 21.44 2θ. Further, the salt of Formula III such as the salt of Formula Va may be characterized by an XRP diffractogram comprising a peak at about 15.27 2θ, and optionally at least one further peak selected from the following: about 7.62 such as about 7.6, about 12.98 such as about 13.0, about 21.84 such as about 21.8, about 22.98 such as about 23.0 2θ. The salt of Formula Va may also be characterized by an XRP diffractogram comprising a peak at about 7.62, about 12.98, about 15.27, about 21.84, about 22.98 2θ, and optionally at least one further peak selected from the following: about 18.55, about 24.08 2θ. For instance, the XRP diffractogram may comprise peaks at about 7.6, about 13.0, about 15.3, about 21.8 and about 23.0 2θ. The salt of Formula Va may also be characterized by an XRP diffractogram comprising a peak at about 7.62, about 12.98, about 15.27, about 18.55, about 21.84, about 22.98, about 24.08 2θ, and optionally at least one further peak selected from the following: about 22.65, about 30.79 20.

The salt of Formula III such as the salt of Formula IV and the salt of Formula V has been found to have a high melting point and a satisfactory water solubility. The high melting point of the salt of Formula III is a benefit in, for instance, tablet making. The satisfactory water solubility of the salt of Formula III makes it suitable for any administration to a human, such as oral administration. The salt of Formula IVa has been found to have a melting point of about 187.6° C. Further, the water solubility of the salt of Formula IVa has been found to be about 185 mg/mL. The salt of Formula Va has been found to have a melting point of about 184.9° C. Further, the water solubility of the salt of Formula Va has been found to be about 92 mg/mL. The melting point and/or water solubility may be determined as described in the Examples section of this document.

Furthermore, the salt of Formula IV has been found to not being hygroscopic at any tested relative humidity and that is advantageous since it allows for storage without being changed by surrounding humidity. It has been found that the salt of Formula IV changes its weight by ±0.3% by weight or less at any humidity such as any relative humidity as described herein, i.e. it is not hygroscopic or substantially not hygroscopic. In an example, the salt of Formula IV does not change its weight at any humidity such as any tested relative humidity.

The advantageous properties of the salt of Formula III with respect to crystallinity, solubility and/or hygroscopicity makes it suitable for incorporation into the pharmaceutical composition described herein. Further, it has unexpectedly been found that the presence of the salt of Formula III in the pharmaceutical composition prevents the L-DOPA sensitization process that allows for treating Parkinson's disease without concomitant inducement of dyskinesias. As a consequence, the dosage of the pharmaceutical drug for Parkinson's disease such as L-DOPA can be adjusted to overcome the symptoms of the patient without being limited by emerging dyskinesias. Further, the pharmaceutical composition described herein prevents L-DOPA sensitization which is advantageous for all patients, in particular patients with an increased risk for developing dyskinesias. Thus, in one embodiment the present invention provides a compound of Formula I or a pharmaceutically acceptable salt thereof (such as the salt of Formula III, preferably the salt of Formula IV, e.g. the salt of Formula IVa) for use in optimization of the dosage of a pharmaceutical drug for Parkinson's disease. The present invention further provides a compound of Formula I or a pharmaceutically acceptable salt thereof (such as the salt of Formula III, preferably the salt of Formula IV, e.g. the salt of Formula IVa) for the manufacture of a medicament for use in optimization of the dosage of a pharmaceutical drug for Parkinson's disease. The present invention also provides a method for the optimization of the dosage of a pharmaceutical drug for Parkinson's disease, said method comprising administering to a subject (e.g. a patient such as a human) in need thereof a therapeutically effective amount of a compound of Formula I or a pharmaceutically acceptable salt thereof (such as the salt of Formula III, preferably the salt of Formula IV, e.g. the salt of Formula IVa).

In certain embodiments relating to optimization of the dosage of a pharmaceutical drug for Parkinson's disease, the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease. Preferably the pharmaceutical drug for Parkinson's disease comprises or consists of L-DOPA or a pharmaceutically acceptable salt thereof.

Optimization of the dosage may comprise administering a dosage of the drug for Parkinson's disease (e.g. L-DOPA or a pharmaceutically acceptable salt thereof) which provides optimal treatment of the motor symptoms associated with Parkinson's disease. Such dosages are often avoided or are reduced after initial administration due to their association with dyskinesias; however, in view of the prevention or reduction of sensitization which is provided by the present invention, such dosages can be administered in order to achieve optimal treatment of motor symptoms without inducing dyskinesias to the same extent as would otherwise be observed. In an embodiment, optimization of the dosage may comprise increasing the dosage of the pharmaceutical drug for Parkinson's disease (e.g. L-DOPA or a pharmaceutically acceptable salt thereof) which is administered to a subject over time. Thus, in an embodiment the present invention provides a compound of Formula I or a pharmaceutically acceptable salt thereof (such as the salt of Formula III, preferably the salt of Formula IV, e.g. the salt of Formula IVa) for use in optimization of the dosage of a pharmaceutical drug for Parkinson's disease (e.g. L-DOPA or a pharmaceutically acceptable salt thereof) wherein optimization of the dosage comprises administering the pharmaceutical drug for Parkinson's disease to a subject in an amount which is increased over time. The present invention further provides a method for the optimization of the dosage of a pharmaceutical drug for Parkinson's disease, said method comprising administering to a subject in need thereof a therapeutically effective amount of a compound of Formula I or a pharmaceutically acceptable salt thereof (such as the salt of Formula III, preferably the salt of Formula IV, e.g. the salt of Formula IVa) and wherein optimization of the dosage comprises administering the pharmaceutical drug for Parkinson's disease (e.g. L-DOPA or a pharmaceutically acceptable salt thereof) to the subject in an amount which is increased over time.

There is also provided a pharmaceutical composition as described herein, wherein the salt of Formula III is characterized by an XRP diffractogram comprising a peak at 13.0 2θ and one or more peaks selected from the following: 12.4, 14.4, 21.1, 24.4 2θ.

The pharmaceutical composition described herein may be provided as a single composition. The single composition may be provided as a tablet, cachet or capsule. The components in the single composition may be mixed together, such as homogenously mixed together.

Further, the pharmaceutical composition described herein may provided as a kit of parts comprising:

(i), (ii) and (iii) as described herein, and
(iv) optionally instructions for use.

The components (i) and/or (ii) may be provided in admixture with (iii). Further, the components (i) and (ii) may independently be provided as a tablet, cachet or capsule.

It will be appreciated that the pharmaceutical composition described herein may comprise a pharmaceutical drug for Parkinson's disease other than L-DOPA or in addition to the L-DOPA. This pharmaceutical drug for Parkinson's disease may be selected from the group consisting of: apomorphine, a derivative of L-DOPA, pramipexole, ropinirole, rotigotine, and a pharmaceutically acceptable salt of any one of the foregoing pharmaceutical drugs for Parkinson's disease. Further, it will be appreciated that the salt of Formula III in the pharmaceutical composition described herein may be replaced with another pharmaceutically acceptable salt of the compound of Formula I or replaced with the compound of Formula I.

The present disclosure also provides a pharmaceutical composition as described herein for use in the treatment and/or prevention of Parkinson's disease.

Further, there is provided a pharmaceutical composition as described herein, wherein the treatment and/or prevention further comprises prevention of sensitization to the drug for Parkinson's disease such as prevention of the sensitization to the L-DOPA or salt thereof.

This prevention of sensitization is advantageous since it minimizes the risk for development of dyskinesias such as LIDs. Unexpectedly, the pharmaceutical composition described herein has been found to prevent sensitization to the drug for Parkinson's disease such as prevention of the sensitization to the L-DOPA or salt thereof. In other words, the pharmaceutical composition described herein has been found to minimize or avoid sensitization to the drug for Parkinson's disease such as prevention of the sensitization to the L-DOPA or salt thereof.

Thus, in one embodiment, the present invention provides a compound of Formula I or a pharmaceutically acceptable salt thereof (such as the salt of Formula III, preferably the salt of Formula IV, e.g. the salt of Formula IVa) for use in the prevention or reduction of sensitization to a pharmaceutical drug for Parkinson's disease. In certain embodiments the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease. Preferably the pharmaceutical drug for Parkinson's disease comprises or consists of L-DOPA or a pharmaceutically acceptable salt thereof. Thus, in an embodiment, prevention or reduction of sensitization comprises or consists of prevention or reduction of L-DOPA sensitization.

The present invention also provides a compound of Formula I or a pharmaceutically acceptable salt thereof (such as the salt of Formula III, preferably the salt of Formula IV, e.g. the salt of Formula IVa) for the manufacture of a medicament for use in the prevention or reduction of sensitization to a pharmaceutical drug for Parkinson's disease. In certain embodiments the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease. Preferably the pharmaceutical drug for Parkinson's disease comprises or consists of L-DOPA or a pharmaceutically acceptable salt thereof. Thus, in an embodiment, prevention or reduction of sensitization comprises or consists of prevention or reduction of L-DOPA sensitization.

The present invention also provides a method for the prevention or reduction of sensitization to a pharmaceutical drug for Parkinson's disease, said method comprising administering to a subject in need thereof (e.g. a patient such as a human) a therapeutically effective amount of a compound of Formula I or a pharmaceutically acceptable salt thereof (such as the salt of Formula III, preferably the salt of Formula IV, e.g. the salt of Formula IVa). In certain embodiments the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease. Preferably the pharmaceutical drug for Parkinson's disease comprises or consists of L-DOPA or a pharmaceutically acceptable salt thereof. Thus, in an embodiment, prevention or reduction of sensitization comprises or consists of prevention or reduction of L-DOPA sensitization.

Further, there is provided a pharmaceutical composition for use as described herein, wherein the pharmaceutical composition is administered before dyskinesias have occurred. Additionally, or alternatively, the pharmaceutical composition may be administered without being preceded by administration of a pharmaceutical drug for Parkinson's disease. Thus, the pharmaceutical composition may be administered without precedent administration of a pharmaceutical drug for Parkinson's disease such as L-DOPA or a pharmaceutically acceptable salt thereof.

Thus, in an embodiment of the invention, the compound of Formula I or pharmaceutically acceptable salt thereof (such as the salt of Formula III, preferably the salt of Formula IV, e.g. the salt of Formula IVa) is administered to a subject who has not experienced dyskinesias, e.g. a subject who has not experienced L-DOPA induced dyskinesias. Such subjects may, for example, be patients (e.g. human patients) who are already undergoing a course of treatment with a pharmaceutical drug for Parkinson's disease, e.g. with L-DOPA or a pharmaceutically acceptable salt thereof, but who have not yet exhibited dyskinetic symptoms (e.g. LIDs). Thus, in certain embodiments, the compound of Formula I or pharmaceutically acceptable salt thereof (such as the salt of Formula III, preferably the salt of Formula IV, e.g. the salt of Formula IVa) is administered to a subject (preferably a human subject) who has been undergoing a course of treatment with a pharmaceutical drug for Parkinson's disease (e.g. with L-DOPA or a pharmaceutically acceptable salt thereof) for at least 1 day, at least 1 week, at least 1 month or at least 1 year (e.g. at least 4, 5, 6, 7, 8, 9 or 10 years) prior to first being administered with the compound of Formula I or pharmaceutically acceptable salt thereof. In another embodiment of the invention, a subject who has not experienced dyskinesias may be a subject who has not previously been administered a pharmaceutical drug for Parkinson's disease, i.e. a subject (e.g. a human patient) who has not undergone or is not undergoing a course of treatment with a pharmaceutical drug for Parkinson's disease (e.g. L-DOPA or a pharmaceutically acceptable salt thereof) prior to commencing treatment with the compound of Formula I or pharmaceutically acceptable salt thereof. In an embodiment, such a subject may undergo “pre-treatment” with the compound of Formula I or pharmaceutically acceptable salt thereof (such as the salt of Formula III, preferably the salt of Formula IV, e.g. the salt of Formula IVa) prior to commencing a course of treatment with a pharmaceutical drug for Parkinson's disease, such as L-DOPA or a pharmaceutically acceptable salt thereof. Thus, the compound of Formula I or pharmaceutically acceptable salt thereof may be administered to the subject (e.g. a human subject) at least 1 day, at least 1 week, at least 1 month or at least 1 year prior to the subject being administered with a pharmaceutical drug for the treatment of Parkinson's disease (e.g. L-DOPA or a pharmaceutically acceptable salt thereof) for the first time. In such embodiments the subject may thus undergo a course of “pre-treatment” in which the compound of Formula I or pharmaceutically acceptable salt thereof is repeatedly administered to the subject e.g. on a daily or twice-daily basis for at least 1 day, at least 1 week, at least 1 month or at least 1 year, prior to being administered with a pharmaceutical drug for the treatment of Parkinson's disease for the first time. In another embodiment, the subject may commence treatment with a pharmaceutical drug for Parkinson's disease (e.g. L-DOPA or a pharmaceutically acceptable salt thereof) on the same day that they also commence treatment with the compound of Formula I or pharmaceutically acceptable salt thereof (such as the salt of Formula III, preferably the salt of Formula IV, e.g. the salt of Formula IVa). Thus, the subject may be administered with (i) the compound of Formula I or pharmaceutically acceptable salt thereof and (ii) the pharmaceutical drug for Parkinson's disease such that the subject receives their first doses of both the compound of Formula I or pharmaceutically acceptable salt thereof and the pharmaceutical drug for Parkinson's disease within 24 hours of one another. In such an embodiment the compound of Formula I or pharmaceutically acceptable salt thereof and the pharmaceutical drug for Parkinson's disease may be administered simultaneously, or the compound of Formula I or pharmaceutically acceptable salt thereof may be administered before the pharmaceutical drug for Parkinson's disease, or the pharmaceutical drug for Parkinson's disease may be administered before the compound of Formula I or pharmaceutically acceptable salt thereof.

The components of the pharmaceutical composition as described herein may be administered, such as administered to a patient simultaneously. Thus, there is provided a pharmaceutical composition for use as described herein, wherein (i) and (ii) are administrated simultaneously.

Alternatively, the components of the pharmaceutical composition described herein may be administered, such as administered to a patient, separately. For instance, (i) may be administered before (ii) or vice versa. Further, (i) and/or (ii) may be provided in admixture with (iii), i.e. a pharmaceutically acceptable excipient, carrier and/or diluent. Thus, there is provided a pharmaceutical composition for use as described herein, wherein

(i) is administrated before (ii), or
(ii) is administrated before (i).

The simultaneous or separate administration of (i) and (ii) may take place within a time period from about 1 second to about 24 hours, such as from about 1 minute to about 24 hours, such as from about 1 minute to about 12 hours, such as from about 1 minute to about 6 hours, such as from about 1 minute to about 1 hour.

Thus the compound of Formula I or pharmaceutically acceptable salt thereof (such as the salt of Formula III, preferably the salt of Formula IV, e.g. the salt of Formula IVa) may be administered to a subject simultaneously with a pharmaceutical drug for Parkinson's disease (e.g. L-DOPA or a pharmaceutically acceptable salt thereof). Alternatively the compound of Formula I or pharmaceutically acceptable salt thereof (such as the salt of Formula III, preferably the salt of Formula IV, e.g. the salt of Formula IVa) may be administered to a subject before a pharmaceutical drug for Parkinson's disease (e.g. L-DOPA or a pharmaceutically acceptable salt thereof), or a pharmaceutical drug for Parkinson's disease (e.g. L-DOPA or a pharmaceutically acceptable salt thereof) may be administered to a subject before the compound of Formula I or pharmaceutically acceptable salt thereof (such as the salt of Formula III, preferably the salt of Formula IV, e.g. the salt of Formula IVa). Where the compound of Formula I or pharmaceutically acceptable salt thereof (such as the salt of Formula III, preferably the salt of Formula IV, e.g. the salt of Formula IVa) is administered before the pharmaceutical drug for Parkinson's disease (e.g. L-DOPA or a pharmaceutically acceptable salt thereof), or vice versa, the administration of the compound of Formula I or pharmaceutically acceptable salt thereof and the administration of the pharmaceutical drug for Parkinson's disease may take place within a time period of from 1 second to 24 hours, such as from about 1 minute to about 24 hours, such as from about 1 minute to about 12 hours, such as from about 1 minute to about 6 hours, such as from about 1 minute to about 1 hour.

Advantageously, the pharmaceutical composition described herein may be administered to a patient after the patient has been diagnosed with Parkinson's disease such as diagnosed with Parkinson's disease by a health care professional such as a physician. Thus, there is provided a pharmaceutical composition for use as described herein, wherein the pharmaceutical composition is administered after diagnosis with Parkinson's disease.

There is also provided a use of a pharmaceutical composition as described herein for the manufacture of a medicament for use in the treatment and/or prevention of Parkinson's disease. The treatment and/or prevention may further comprise preventing sensitization to a pharmaceutical drug for Parkinson's disease such as L-DOPA or a pharmaceutically acceptable salt thereof. The pharmaceutical composition may be administered before dyskinesias have occurred. Additionally or alternatively, the pharmaceutical composition may be administered without being preceded by administration of a pharmaceutical drug for Parkinson's disease such as L-DOPA or a pharmaceutically acceptable salt thereof. Further, (i) and (ii) of the pharmaceutical composition may be administrated simultaneously or separately. In the latter case, (i) may be administrated before (ii), or (ii) may be administrated before (i). Further, (i) and (ii) may be administrated within a time period of from about 1 second to about 24 hours, such as from 1 about minute to about 24 hours, such as from about 1 minute to about 12 hours, such as from about 1 minute to about 6 hours, such as from about 1 minute to about 1 hour. Moreover, the pharmaceutical composition may be administered to a patient (preferably a human subject)

who has been diagnosed with Parkinson's disease, and/or
who has one or more following characteristics:
being below the age of about 60 years,
being a woman,
exhibiting severe motor and functional impairment,
with a genetic susceptibility for Parkinson's disease,
suffering from anxiety.

There is also provided a method for treatment and/or prevention of Parkinson's disease, said method comprising administering to a patient, such as a human or an animal, a therapeutically effective amount of the pharmaceutical composition as described herein. The treatment and/or prevention may further comprise prevention of sensitization to a pharmaceutical drug for Parkinson's disease such as L-DOPA or a pharmaceutically acceptable salt thereof. The pharmaceutical composition may be administered before dyskinesias have occurred. Additionally or alternatively, the pharmaceutical composition may be administered without being preceded by administration of a pharmaceutical drug for Parkinson's disease such as L-DOPA or a pharmaceutically acceptable salt thereof. Further, (i) and (ii) of the pharmaceutical composition may be administrated simultaneously or separately. In the latter case, (i) may be administrated before (ii), or (ii) may be administrated before (i). Further, (i) and (ii) may be administrated within a time period of from about 1 second to about 24 hours, such as from about 1 minute to about 24 hours, such as from about 1 minute to about 12 hours, such as from about 1 minute to about 6 hours, such as from about 1 minute to about 1 hour. Moreover, the pharmaceutical composition may be administered to a patient (preferably a human)

who has been diagnosed with Parkinson's disease, and/or
who has one or more following characteristics:
being below the age of about 60 years,
being a woman,
exhibiting severe motor and functional impairment,
with a genetic susceptibility for Parkinson's disease,
suffering from anxiety.

The amount of the compound of Formula I or the salt of Formula III in the pharmaceutical composition described herein may vary. For instance, the amount of the compound of Formula I of said salt may be from about 2.0 mg up to about 10.0 mg such as about 2.5 mg to about 7.5 mg. Further, the amount of the compound of Formula I of said salt may be about 5.0 mg, about 7.5 mg or about 2.5 mg. Thus, in certain embodiments, the compound of Formula I or a pharmaceutically acceptable salt thereof (such as the salt of Formula III, preferably the salt of Formula IV, e.g. the salt of Formula IVa) may be administered in an amount corresponding to about 2.0 mg up to about 10.0 mg of the compound of Formula I, e.g. an amount corresponding to about 2.5 mg to about 7.5 mg of the compound of Formula I, an amount corresponding to about 7.5 mg up to about 10.0 mg of the compound of Formula I, an amount corresponding to about 2.5 mg to about 5.0 mg of the compound of Formula I, an amount corresponding to about 2.5 mg of the compound of Formula I, an amount corresponding to about 5.0 mg of the compound of Formula I, or an amount corresponding to about 7.5 mg of the compound of Formula I.

It has been found that administration of the aforementioned amounts to a patient such as a human reduces L-DOPA induced dyskinesias, to a greater extent as compared to administration of the compound of Formula I or the salt of Formula III in a higher amount such as an amount equal to or above about 10 mg. Thus, administration of a lower amount is more advantageous than administration of a higher amount.

In all embodiments of the invention as described herein, the compound of Formula I or pharmaceutically acceptable salt thereof (such as the salt of Formula III, preferably the salt of Formula IV, e.g. the salt of Formula IVa) may if desired be administered twice per day, e.g. in twice daily dosages each corresponding to one of the aforementioned amounts. Such dosages may, for example, be given once in the morning and once in the evening. In an embodiment the compound of Formula I or pharmaceutically acceptable salt thereof may be administered in two equal daily dosages, e.g. two dosages per day each corresponding to about 7.5 mg, such as about 7.5 mg in the morning and about 7.5 mg in the evening; or two dosages per day each corresponding to about 5.0 mg, such as about 5.0 mg in the morning and about 5.0 mg in the evening; or two dosages per day each corresponding to about 2.5 mg such as about 2.5 mg in the morning and 2.5 mg in the evening.

In all embodiments of the invention as described herein, the compound of Formula I or pharmaceutically acceptable salt thereof (such as the salt of Formula III, preferably the salt of Formula IV, e.g. the salt of Formula IVa) may if desired be administered in a total daily dosage of from about 4.0 mg up to about 20.0 mg, e.g. in a total daily dosage of about 5.0 mg to about 15.0 mg, such as about 5.0 mg to about 10.0 mg.

Without wishing to be bound by theory, the present inventors believe that the compound of Formula I and pharmaceutically acceptable salts thereof as described herein block the sensitization process which is responsible for the development of dyskinesias such as LIDs and that this blocking effect is most pronounced in the population of neurons which are most susceptible to sensitization. Thus, the compound of Formula I or pharmaceutically acceptable salt thereof may advantageously be administered in one or more doses in a first (higher) amount, e.g. for a first time period such as an initial phase of treatment (such as a period of from at least one week, at least two weeks, or at least one month, up to about 2, 3, 4, 5 or 6 months), and then administered in one or more doses in a second (lower) amount, e.g. for a second time period such as a longer-term phase of treatment (e.g. with a lower, “maintenance” dose, for example for a period of at least 6 months or at least 1, at least about 2, at least 3, at least 4, or at least 5 years). Such phases of treatment may each typically comprise administration (e.g. repeated administration) of discrete doses of a particular amount of the compound of Formula I or pharmaceutically acceptable salt thereof to a subject. Thus, in an embodiment of the invention, the compound of Formula I or pharmaceutically acceptable salt thereof (such as the salt of Formula III, preferably the salt of Formula IV, e.g. the salt of Formula IVa) may initially be administered in one or more doses in a first amount, e.g. a dose corresponding to about 7.5 mg up to about 10.0 mg of the compound of Formula I, and then administered in one or more doses in a second amount, wherein said second amount is lower than said first amount (e.g. wherein the second amount is a dose corresponding to about 2.5 to about 5.0 mg of the compound of Formula I). In an illustrative embodiment, the compound of Formula I or pharmaceutically acceptable salt thereof may be administered in two doses per day in a first amount, e.g. in two doses per day each of about 7.5 mg, and then in two doses per day in a second amount which is lower than said first amount, e.g. in two doses per day each of about 5.0 mg or in two doses per day each of about 2.5 mg. In an embodiment, the compound of Formula I or pharmaceutically acceptable salt thereof may be administered in one or more doses in a first amount such as administered in a total daily dosage of about 15.0 mg to about 20.0 mg, and then administered in one or more doses in a second amount such as administered in a total daily dosage of about 4.0 mg to 10.0 mg, e.g. a total daily dosage of about 5.0 mg to about 10.0 mg.

In an embodiment, the administration of one or more doses in a first amount and the administration of one or more doses in a second amount may take place during, respectively, a first time period and a second time period, wherein said first time period and said second time period are independently at least one day, at least one week, at least one month or at least one year. During such first and second time periods, the compound of Formula I or pharmaceutically acceptable salt thereof may be administered (e.g. repeatedly administered) in discrete doses. In an illustrative embodiment, the compound of Formula I or pharmaceutically acceptable salt thereof may be administered in one or more doses in a first amount (e.g. administered in two doses per day each of about 7.5 mg, or administered in a total daily dosage of about 15.0 mg), for a first time period such as a period of from two weeks to two months, and then in one or more doses in a second amount (e.g. administered in two doses per day each of about 5.0 mg or each of about 2.5 mg, or administered in a total daily dosage of about 10.0 mg or about 5.0 mg) for a second time period such as a period of at least six months. In certain embodiments the first time period may correspond to a period of “pre-treatment” during which the subject receives the compound of Formula I or pharmaceutically acceptable salt thereof (such as the salt of Formula III, preferably the salt of Formula IV, e.g. the salt of Formula IVa) without coadministration of a pharmaceutical drug for Parkinson's disease and the second time period may correspond to a period during which the subject is co-administered the compound of Formula I or pharmaceutically acceptable salt thereof and a pharmaceutical drug for Parkinson's disease (e.g. L-DOPA or a pharmaceutically acceptable salt thereof). Thus in an embodiment of the invention the compound of Formula I or pharmaceutically acceptable salt thereof is administered in one or more doses in a first amount without coadministration of a pharmaceutical drug for Parkinson's disease, and then administered in one or more doses in a second amount with coadministration of a pharmaceutical drug for Parkinson's disease, wherein said second amount is lower than said first amount. In an embodiment, the subject may be co-administered the compound of Formula I or pharmaceutically acceptable salt thereof (such as the salt of Formula III, preferably the salt of Formula IV, e.g. the salt of Formula IVa) and a pharmaceutical drug for Parkinson's disease (e.g. L-DOPA or a pharmaceutically acceptable salt thereof) during both the first and the second time periods. Thus in an embodiment of the invention the compound of Formula I or pharmaceutically acceptable salt thereof is administered in one or more doses in a first amount with coadministration of a pharmaceutical drug for Parkinson's disease, and then administered in one or more doses in a second amount with coadministration of a pharmaceutical drug for Parkinson's disease, wherein said second amount is lower than said first amount. In an embodiment the pharmaceutical drug for Parkinson's disease may be administered in a dosage which is increased when the compound of Formula I or pharmaceutically acceptable salt thereof is administered in the second amount.

Co-administration of the compound of Formula I or pharmaceutically acceptable salt thereof and the pharmaceutical drug for Parkinson's disease may take place simultaneously or may take place sequentially as described herein, i.e. with administration of the compound of Formula I preceding administration of the pharmaceutical drug for Parkinson's disease or vice versa. Thus, the compound of Formula I or pharmaceutically acceptable salt thereof and the pharmaceutical drug for Parkinson's disease may be administrated within a time period of from about 1 second to about 24 hours, such as from about 1 minute to about 24 hours, such as from about 1 minute to about 12 hours, such as from about 1 minute to about 6 hours, such as from about 1 minute to about 1 hour. As used herein, the expression “from . . . up to . . . ” intends “from . . . up to but not including . . . ”. For instance, the expression “from 2.0 mg up to 10.0 mg” intends “from 2.0 mg up to but not including 10.0 mg”. In the latter case, the amount 9.99 mg is included while the amount 10.0 mg is not included.

It will be appreciated that the dosage described herein such as a dosage from about 2.0 mg to about 10.0 mg of the salt of Formula III intends the dosage calculation based on the compound of Formula I, i.e. the compound of Formula I in non-salt form. For example, when the dosage is 7.5 mg this means that an amount of 7.5 mg of the compound of Formula I is provided.

Accordingly, the pharmaceutical composition described herein may be administered-such as administered to a patient (preferably a human subject)—to provide a dosage from about 2.0 mg up to about 10 mg of the salt of Formula III (e.g. the salt of Formula IV, e.g. the salt of Formula IVa) or the compound of Formula I, wherein the dosage corresponds to the amount of the freebase (i.e. the compound of Formula I).

Certain patients are more at risk for developing sensitization to a pharmaceutical drug for Parkinson's disease such as L-DOPA or a pharmaceutically acceptable salt thereof. Examples of such patients include patients with one or more following characteristics:

being below the age of about 60 years,
being a woman,
exhibiting severe motor and functional impairment,
with a genetic susceptibility for Parkinson's disease,
suffering from anxiety.

Thus, there is provided a compound for use and a pharmaceutical composition as described herein wherein the compound of Formula I or pharmaceutically acceptable salt thereof (such as the salt of Formula III, preferably the salt of Formula IV, e.g. the salt of Formula IVa) or the pharmaceutical composition is administered to a patient (preferably a human subject) with one or more following characteristics:

being below the age of about 60 years,
being a woman,
exhibiting severe motor and functional impairment,
with a genetic susceptibility for Parkinsons's disease,
suffering from anxiety.

The salt of Formula III may be prepared by combining a compound of Formula I as described herein with an acid of Formula II as described herein. The compound of Formula I may be prepared as described herein, as described in WO 2012/143337 and/or using methods known in the art.

Accordingly, the present disclosure also provides a method for preparing a salt of Formula III as described herein such as a salt of Formula IV or a salt of Formula V said method comprising the steps of:
providing a compound of Formula I as described herein and an acid of Formula II as described herein in a ratio of 1:n such as a ratio of 1:0.5 or 1:1,
combining said compound of Formula I with said acid of Formula II in a solvent to form a solution, and
allowing said solution to stand until a precipitate forms, and
isolating said precipitate by filtration thereby providing the salt of Formula III.

In the method for preparing a salt of Formula III as described herein, the ratio of the compound of Formula I to the acid of Formula II may be 1:0.5 or 1:1. Further, the solvent may be a single solvent or a mixture of solvents. The solvent or mixture of solvents may comprise or consist of organic solvent(s) such as ethanol. Further, the step of forming a precipitate may be performed at room temperature. In this document, room temperature intends a temperature within the range of from about 20° C. to about 25° C., such as from about 20° C. to about 22° C. The acid of Formula II may be tartaric acid or fumaric acid.

It will be appreciated that the pharmaceutical composition described herein may be administered to a patient (preferably a human subject) once daily or several times daily. In the latter case, the administration may take place twice daily, three times daily or four times daily.

Further, it will be appreciated that the pharmaceutical composition described herein optionally may comprise one or more co-administration agents that are used in clinical practice together with L-DOPA such as for instance a peripheral DOPA decarboxylase inhibitor (DDCI).

Salts

In this document, the chemical structure of the salt of Formula III comprising a combination of the compound of Formula I and an acid of Formula II has been drawn as a complex wherein the acidic proton(s) of the acid is attached to said acid. However, the skilled person understands that the acidic proton(s) of the acid of Formula II may be attached to the nitrogen atom of the compound of Formula I and/or shared between the nitrogen atom of the compound of Formula I and the acid of Formula II, and this is also intended to be encompassed by the salts described herein. For instance, the salt of Formula III being a 1:1 combination of the compound of Formula I and the acid of Formula II may also be represented as:

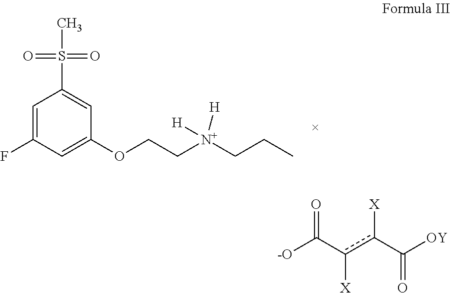

It will be appreciated that the salts of Formula III described herein may be converted to another salt of Formula III using standard procedures known in the art.

Isotopes

The compound of Formula I of the salt of Formula III of the present disclosure may contain an atomic isotope at one or more of the atoms that constitute said compounds, i.e. said compound may be labelled with an isotope. For example, the compound of Formula I may be labelled with one or more isotopes, such as for example tritium (³H), deuterium (²H), iodine-125 (¹²⁵I) or carbon-14 (¹⁴O). In an example, the compound is labelled with one or more deuterium atoms. All isotopic variations of the compounds of the present disclosure, whether radioactive or not, are intended to be encompassed within the scope of the present disclosure.

Thus, the present disclosure provides a compound as described herein, such as a compound of Formula I, which is labelled with one or more isotopes such as deuterium. The compounds labelled with an isotope as described herein may be combined with an acid as described herein thereby providing a salt as described herein.

The present disclosure also provides the following items.

Items
1. A pharmaceutical composition comprising:
   (i) a salt of Formula III:

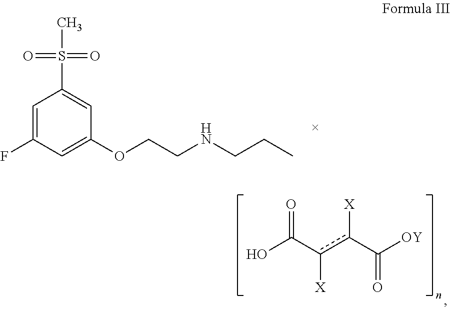

said salt being a combination of a compound of Formula I and an acid of Formula II:

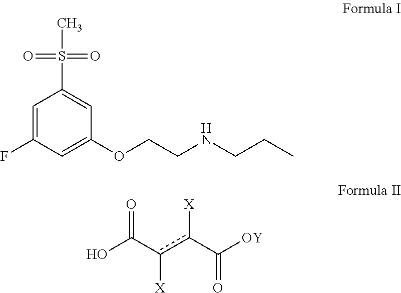

in a ratio of 1:n,
wherein
X is H or OH,
Y is H or a cation selected from the group consisting of Li, Na and K,
[symbol] is a single bond or
a double bond, and
n is 0.5 or 1,
(ii) a pharmaceutical drug for Parkinson's disease or a pharmaceutically acceptable salt thereof, and
(iii) optionally a pharmaceutically acceptable excipient, carrier and/or diluent.
2. The pharmaceutical composition according to item 1, wherein the following values apply for the salt of Formula III:
   X is OH,
   Y is H, and
   [symbol] is a single bond,
   thereby providing a salt of Formula IV being a combination of a compound of Formula I and tartaric acid:

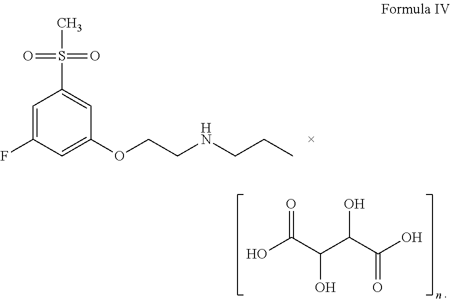

3. The pharmaceutical composition according to item 1 or 2, wherein the acid of Formula II is tartaric acid such as L-(+)-tartaric acid and/or D-(−)-tartaric acid.
4. The pharmaceutical composition according to item 1, wherein the following values apply for the salt of Formula III:
   X is H,
   Y is H, and
   [symbol] is a double bond,
   thereby providing a salt of Formula V being a combination of a compound of Formula V and fumaric acid:

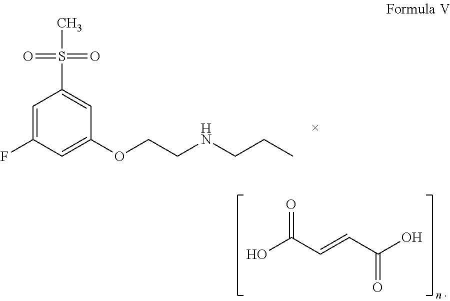

5. The pharmaceutical composition according to item 1, wherein n is 0.5.
6. The pharmaceutical composition according to item 1, wherein n is 1.
7. The pharmaceutical composition according to any one of the preceding items, wherein one or more of the hydrogen atoms of the compound of Formula I is/are replaced with deuterium.
8. The pharmaceutical composition according to any one of the preceding items, wherein the salt of Formula III is crystalline.
9. The pharmaceutical composition according to
   any one of items 1-3, 5 or 7, wherein the salt of Formula III is characterized by an XRP diffractogram as shown in FIG. 1, or
   any one of items 1, 4, 5 or 7, wherein the salt of Formula III is characterized by an XRP diffractogram as shown in FIG. 2.
10. The pharmaceutical composition according to any one of items 1-3, 5 or 7, wherein the salt of Formula III is characterized by an XRP diffractogram comprising a peak at 13.0 2θ and one or more peaks selected from the following:
   12.4, 14.4, 21.1, 24.4 2θ.
11. The pharmaceutical composition according to any one of items 1, 4, 5 or 7, wherein the salt of Formula III is characterized by an XRP diffractogram comprising a peak at 15.3 20 and one or more peaks selected from the following: 7.6, 13.0, 21.8, 23.0 2θ.
12. The pharmaceutical composition according to any one of the preceding items, wherein the compound of Formula I or the salt of Formula III is present in an amount from about 2.0 mg up to about 10.0 mg.
13. The pharmaceutical composition according to any one of the preceding items, wherein the amount of the compound of Formula I or the salt of Formula III is about 2.5 mg, about 5.0 mg or about 7.5 mg.
14. The pharmaceutical composition according to any one of the preceding items, wherein the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease.
15. The pharmaceutical composition according to any one of the preceding items, wherein the pharmaceutical drug for Parkinson's disease comprises or consists of L-DOPA or a pharmaceutically acceptable salt thereof.
16. The pharmaceutical composition according to any one of the preceding items, for use in the treatment and/or prevention of Parkinson's disease.
17. The pharmaceutical composition for use according to item 16, wherein the treatment and/or prevention further comprises prevention of sensitization to the pharmaceutical drug for Parkinson's disease.
18. The pharmaceutical composition for use according to item 16 or 17, wherein the treatment and/or prevention further comprises prevention of sensitization to L-DOPA, i.e. prevention of L-DOPA sensitization.
19. The pharmaceutical composition for use according to any one of items 16-18, wherein the treatment and/or prevention further comprises prevention of dyskinesias such as prevention of L-DOPA induced dyskinesias.
20. The pharmaceutical composition for use according to any one of items 16-19, wherein the treatment and/or prevention further comprises prevention of L-DOPA induced dyskinesias.
21. The pharmaceutical composition for use according to items 16-20, wherein the pharmaceutical composition is administered before dyskinesias have occurred.
22. The pharmaceutical composition for use according to items 16-21, wherein the pharmaceutical composition is administered without precedent administration of a pharmaceutical drug for Parkinson's disease such as L-DOPA.
23. The pharmaceutical composition for use according to any one of items 16-22, wherein (i) and (ii) are administrated simultaneously.
24. The pharmaceutical composition for use according to any one of items 16-22, wherein
   (i) is administrated before (ii), or
   (ii) is administrated before (i).
25. The pharmaceutical composition for use according to any one of items 16-24, wherein (i) and (ii) are administrated within a time period of from about 1 second to about 24 hours, such as from about 1 minute to about 24 hours, such as from about 1 minute to about 12 hours, such as from about 1 minute to about 6 hours, such as from about 1 minute to about 1 hour.
26. The pharmaceutical composition for use according to any one of items 16-25, wherein the pharmaceutical composition is administered, such as administered to a patient after diagnosis with Parkinson's disease.
27. The pharmaceutical composition for use according to any one of items 16-26, wherein the pharmaceutical composition is administered to a patient at risk for Parkinson's disease such as a patient with one or more following characteristics:
   being below the age of about 60 years,
   being a woman,
   exhibiting severe motor and functional impairment,
   with a genetic susceptibility for Parkinson's disease,
   suffering from anxiety.
28. The pharmaceutical composition for use according to any one of items 16-27, wherein the pharmaceutical composition is administered to provide a dosage of the compound of Formula I or the salt of Formula III in an amount of from about 2.0 mg up to about 10.0 mg such as about 2.5 mg, about 5.0 mg or about 7.5 mg.
29. Use of a pharmaceutical composition according to any one of items 1-15 for the manufacture of a medicament for the treatment and/or prevention of Parkinson's disease.
30. The use according to item 29, wherein the treatment and/or prevention further comprises prevention of sensitization to the pharmaceutical drug for Parkinson's disease.
31. The use according to item 29 or 30, wherein the treatment and/or prevention further comprises prevention of sensitization to L-DOPA, i.e. prevention of L-DOPA sensitization.
32. The use according to any one of items 29-31, wherein the treatment and/or prevention further comprises prevention of dyskinesias, such as prevention of L-DOPA induced dyskinesias.
33. The use according to any one of items 29-32, wherein the treatment and/or prevention further comprises prevention of L-DOPA induced dyskinesias.
34. The use according to any one of items 29-33, wherein the pharmaceutical composition is administered before dyskinesias have occurred.
35. The use according to any one of items 29-34, wherein the pharmaceutical composition is administered without precedent administration of a pharmaceutical drug for Parkinson's disease such as L-DOPA.
36. The use according to any one of items 29-35, wherein (i) and (ii) are administrated simultaneously.
37. The use according to any one of items 29-35, wherein
   (i) is administrated before (ii), or
   (ii) is administrated before (i).
38. The use according to any one of items 29-37, wherein (i) and (ii) are administrated within a time period of from about 1 second to about 24 hours, such as from about 1 minute to about 24 hours, such as from about 1 minute to about 12 hours, such as from about 1 minute to about 6 hours, such as from about 1 minute to about 1 hour.
39. The use according to any one of items 29-38, wherein the pharmaceutical composition is administered after diagnosis with Parkinson's disease.
40. The use according to any one of items 29-39, wherein the pharmaceutical composition is administered to a patient at risk for Parkinson's disease such as a patient with one or more following characteristics:
   being below the age of about 60 years,
   being a woman,
   exhibiting severe motor and functional impairment,
   with a genetic susceptibility for Parkinson's disease,
   suffering from anxiety.
41. The use according to any one of items 29-40, wherein the pharmaceutical composition is administered to provide a dosage of the compound of Formula I or the salt of Formula III in an amount of from about 2.0 mg up to about 10.0 mg such as about 2.5 mg, about 5.0 mg or about 7.5 mg.
42. A method for treatment and/or prevention of Parkinson's disease, the method comprising administering to a patient such as a human in need thereof a therapeutically effective amount of the pharmaceutical composition according to any one of items 1-15.
43. The method according to item 42, wherein the treatment and/or prevention further comprises prevention of sensitization to the pharmaceutical drug for Parkinson's disease.
44. The method according to item 41 or 42, wherein the treatment and/or prevention further comprises prevention of sensitization to L-DOPA, i.e. prevention of L-DOPA sensitization.
45. The method according to any one of items 42-44, wherein the treatment and/or prevention further comprises prevention of dyskinesias, such as prevention of L-DOPA induced dyskinesias.
46. The method according to any one of items 42-45, wherein the treatment and/or prevention further comprises prevention of L-DOPA induced dyskinesias.
47. The method according to any one of items 42-46, wherein the pharmaceutical composition is administered before dyskinesias have occurred.
48. The method according to any one of items 42-47, wherein the pharmaceutical composition is administered without precedent administration of a pharmaceutical drug for Parkinson's disease such as L-DOPA.
49. The method according to any one of items 42-48, wherein (i) and (ii) are administrated simultaneously.
50. The method according to any one of items 42-48 wherein
   (i) is administrated before (ii), or
   (ii) is administrated before (i).
51. The method according to any one of items 42-50, wherein (i) and (ii) are administrated within a time period of from 1 second to 24 hours, such as from about 1 minute to about 24 hours, such as from about 1 minute to about 12 hours, such as from about 1 minute to about 6 hours, such as from about 1 minute to about 1 hour.
52. The method according to any one of items 42-51, wherein the pharmaceutical composition is administered after diagnosis with Parkinson's disease.
53. The method according to any one of items 42-52, wherein the pharmaceutical composition is administered to a patient at risk for Parkinson's disease such as a patient with one or more following characteristics:
   being below the age of about 60 years,
   being a woman,
   exhibiting severe motor and functional impairment,
   with a genetic susceptibility for Parkinson's disease,
   suffering from anxiety.
54. The method according to any one of items 42-53, wherein the pharmaceutical composition is administered to provide a dosage of the compound of Formula I or the salt of Formula III in an amount of from about 2.0 mg up to about 10.0 mg such as about 2.5 mg, about 5.0 mg or about 7.5 mg.
55. A compound of Formula I,

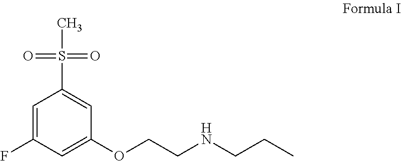 [SMILES: CCCNCCOc1cc(F)cc(S(C)(=O)=O)c1]

or a pharmaceutically acceptable salt thereof,
for use in the prevention of sensitization to a pharmaceutical drug for Parkinson's disease.
56. Use of a compound of Formula I,

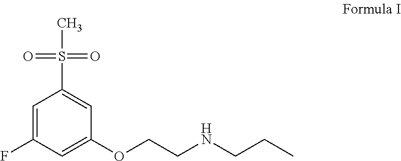 [SMILES: CCCNCCOc1cc(F)cc(S(C)(=O)=O)c1]

or a pharmaceutically acceptable salt thereof,
for the manufacture of a medicament for the prevention of sensitization to a pharmaceutical drug for Parkinson's disease.
57. A method for the prevention of sensitization to a pharmaceutical drug for Parkinson's disease, said method comprising administering to a patient such as a human in need thereof a therapeutically effective amount of a compound of Formula I

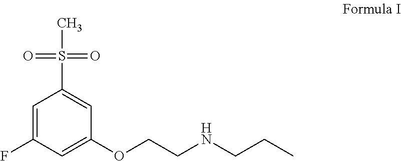 [SMILES: CCCNCCOc1cc(F)cc(S(C)(=O)=O)c1]

or a pharmaceutically acceptable salt thereof.
58. The compound according to item 55,
   the use according to item 56, or
   the method according to item 57
   wherein the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease.
59. The compound according to item 55, or
   the use according to item 56, or
   the method according to item 57
   wherein the pharmaceutical drug for Parkinson's disease comprises or consists of L-DOPA or a pharmaceutically acceptable salt thereof.
60. The compound according to item 55, or
   the use according to item 56, or
   the method according to item 57
   wherein the prevention comprises or consists of sensitization to L-DOPA, i.e. prevention of L-DOPA sensitization.
61. The compound according to item 55, or
   the use according to item 56, or
   the method according to item 57
   wherein no dyskinesias have taken place prior to administration of the compound of Formula I or a pharmaceutically acceptable salt thereof.
62. The compound according to item 55, or
   the use according to item 56, or
   the method according to item 57
   wherein no administration of a pharmaceutical drug for Parkinson's disease, or a pharmaceutically acceptable salt thereof, has taken place prior to administration of the compound of Formula I or a pharmaceutically acceptable salt thereof.
63. The compound according to item 55, or
   the use according to item 56, or
   the method according to item 57
   wherein the pharmaceutically acceptable salt of the compound of Formula I is a salt of Formula III:

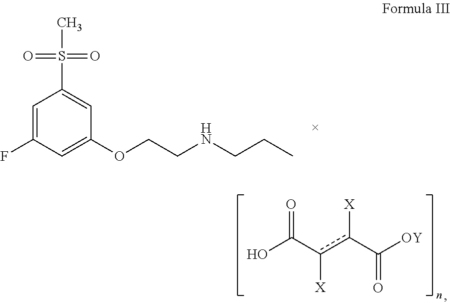

said salt being a combination of a compound of Formula I and an acid of Formula II:

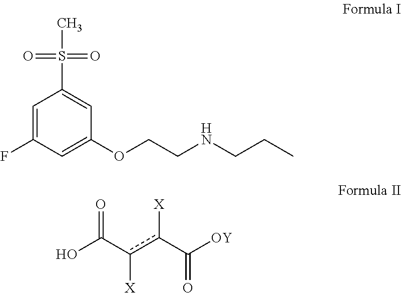

in a ratio of 1:n,
wherein
X is H or OH,
Y is H or a cation selected from the group consisting of Li, Na and K,
[symbol] is a single bond or
a double bond, and
n is 0.5 or 1,
64. The compound according to item 55, or
   the use according to item 56, or
   the method according to item 57
   wherein the compound of Formula I is co-administered, such as separate, sequential or simultaneous co-administration, with a pharmaceutical drug for Parkinson's disease such as L-DOPA or a pharmaceutically acceptable salt thereof.
65. A method for the prevention or reduction of sensitization to a pharmaceutical drug for Parkinson's disease, said method comprising administering to a subject in need thereof a therapeutically effective amount of a compound of Formula I

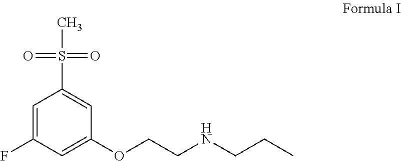 [SMILES: CCCNCCOc1cc(F)cc(S(C)(=O)=O)c1]

or a pharmaceutically acceptable salt thereof.
66. The method of item 65, wherein the pharmaceutical drug for Parkinson's disease is selected from the group consisting of apomorphine, L-DOPA, a derivative of L-DOPA, pramipexole, ropinirole and rotigotine, or a pharmaceutically acceptable salt of any one of the foregoing pharmaceutical drugs for Parkinson's disease.
67. The method of item 65, wherein the pharmaceutical drug for Parkinson's disease comprises or consists of L-DOPA or a pharmaceutically acceptable salt thereof.
68. The method of item 65, wherein prevention or reduction of sensitization to a pharmaceutical drug for Parkinson's disease comprises or consists of prevention or reduction of L-DOPA sensitization.
69. The method of item 65, wherein prevention or reduction of sensitization to a pharmaceutical drug for Parkinson's disease comprises administering the compound of Formula I or a pharmaceutically acceptable salt thereof to a subject who has not experienced dyskinesias.
70. The method of item 65, wherein prevention or reduction of sensitization to a pharmaceutical drug for Parkinson's disease comprises administering the compound of Formula I or a pharmaceutically acceptable salt thereof to a subject who has not experienced L-DOPA induced dyskinesias.
71. The method of item 69, wherein prevention or reduction of sensitization to a pharmaceutical drug for Parkinson's disease comprises administering the compound of Formula I or pharmaceutically acceptable salt thereof to a subject who has been undergoing a course of treatment with a pharmaceutical drug for Parkinson's disease for at least 1 day prior to administration of the compound of Formula I or pharmaceutically acceptable salt thereof to the subject for the first time.
72. The method of item 70, wherein prevention or reduction of sensitization to a pharmaceutical drug for Parkinson's disease comprises administering the compound of Formula I or pharmaceutically acceptable salt thereof to a subject who has been undergoing a course of treatment with L-DOPA or a pharmaceutically acceptable salt thereof for at least 1 day prior to administration of the compound of Formula I or pharmaceutically acceptable salt thereof to the subject for the first time.
73. The method of item 65, wherein prevention or reduction of sensitization to a pharmaceutical drug for Parkinson's disease comprises administering the compound of Formula I or pharmaceutically acceptable salt thereof to a subject who has not previously been administered a pharmaceutical drug for Parkinson's disease.
74. The method of item 73, wherein the subject has not previously been administered L-DOPA or a pharmaceutically acceptable salt thereof.
75. The method of item 73, wherein prevention or reduction of sensitization to a pharmaceutical drug for Parkinson's disease comprises administering the compound of Formula I or pharmaceutically acceptable salt thereof to the subject at least 1 day prior to administration of a pharmaceutical drug for Parkinson's disease to the subject for the first time.
76. The method of item 75, wherein prevention or reduction of sensitization to a pharmaceutical drug for Parkinson's disease comprises administering the compound of Formula I or pharmaceutically acceptable salt therefor to the subject at least 1 day prior to administration of L-DOPA or a pharmaceutically acceptable salt thereof to the subject for the first time.
77. The method of item 65, wherein the compound of Formula I or pharmaceutically acceptable salt thereof is administered to a subject after the subject has been diagnosed with Parkinson's disease.
78. The method of item 65, wherein the compound of Formula I or pharmaceutically acceptable salt thereof is administered to a subject at risk for Parkinson's disease such as a subject with one or more following characteristics:
   being below the age of about 60 years,
   being a woman,
   exhibiting severe motor and functional impairment,
   with a genetic susceptibility for Parkinson's disease,
   suffering from anxiety.
79. The method of item 65, wherein the compound of Formula I or pharmaceutically acceptable salt thereof is administered in an amount corresponding to about 2.0 mg up to about 10.0 mg of the compound of Formula I, such as an amount corresponding to about 2.5 mg, about 5.0 mg or about 7.5 mg of the compound of Formula I.
80. The method of item 65, wherein the compound of Formula I or pharmaceutically acceptable salt thereof is administered in one or more doses in a first amount and then administered in one or more doses in a second amount, wherein said second amount is lower than said first amount.
81. The method of item 80, wherein the first amount corresponds to an amount of about 7.5 up to about 10.0 mg of the compound of Formula I.
82. The method of item 80, wherein the second amount corresponds to an amount of about 2.5 to about 5.0 mg of the compound of Formula I.
83. The method of item 65, wherein the compound of Formula I or pharmaceutically acceptable salt thereof is administered twice per day.
84. The method of item 65, wherein the pharmaceutically acceptable salt of the compound of Formula I is a salt of Formula III:

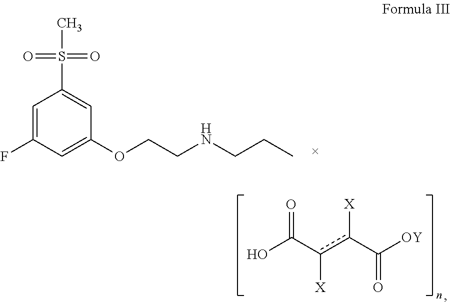

said salt being a combination of a compound of Formula I and an acid of Formula II:

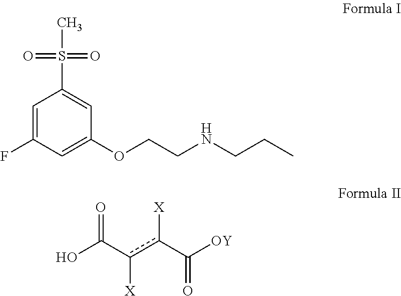

in a ratio of 1:n,
wherein
X is H or OH,
Y is H or a cation selected from the group consisting of Li, Na and K,
[symbol] is a single bond or
a double bond, and
n is 0.5 or 1.
85. The method of item 84, wherein the following values apply for the salt of Formula III:
   X is OH,
   Y is H, and
   [symbol] is a single bond,
   thereby providing a salt of Formula IV being a combination of a compound of Formula I and tartaric acid:

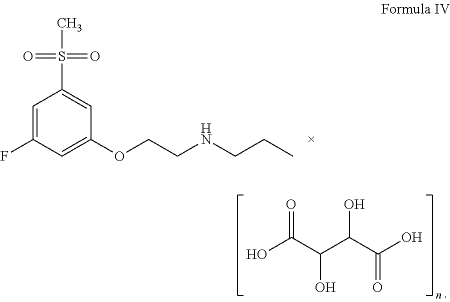

86. The method of item 84, wherein n is 0.5.
87. The method of item 85, wherein n is 0.5.
88. A method for the optimization of the dosage of a pharmaceutical drug for Parkinson's disease, said method comprising administering to a subject in need thereof a therapeutically effective amount of a compound of Formula I

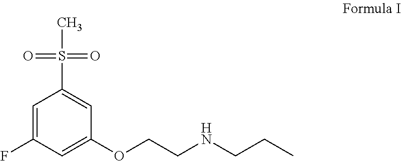 [SMILES: CCCNCCOc1cc(F)cc(S(C)(=O)=O)c1]

or a pharmaceutically acceptable salt thereof.
89. The method of item 88, wherein the pharmaceutical drug for Parkinson's disease is L-DOPA or a pharmaceutically acceptable salt thereof.
90. The method of item 88, wherein optimization of the dosage comprises administering the pharmaceutical drug for Parkinson's disease to the subject in an amount which is increased over time.
91. The method of item 89, wherein optimization of the dosage comprises administering L-DOPA or a pharmaceutically acceptable salt thereof to the subject in an amount which is increased over time.

In all of the foregoing discussion, the skilled person will understand that any features characterizing the compound of Formula I or salts thereof (including pharmaceutically acceptable salts thereof such as the salt of Formula III, and preferably the salt of Formula IV, e.g. the salt of Formula IVa) when present as part of the composition which is disclosed herein may equally be taken to characterize the compound of Formula I per se (including salts thereof including pharmaceutically acceptable salts such as the salt of Formula III, and preferably the salt of Formula IV, e.g. the salt of Formula IVa). This includes any features relating to the dosage and/or administration of the compound of Formula I or salts thereof, e.g. dosage amounts of the compound or characteristics of the subject or patient to whom the compound is administered.

The disclosure is further illustrated by the following non-limiting Examples.

### EXAMPLES

In this document, unless otherwise stated, the drawing of the chemical compounds have been made using the software package Chem Doodle, ver. 9.0.3. The naming of compounds was made using the program MarvinSketch 16.10.17.0. If the drawing and chemical name are inconsistent, the chemical structure shall be considered to be correct.

General

Reagents and solvents were used as purchased without purification.

HPLC analyses were performed on a Dionex HPLC Module with Dionex UVD 170U Detector and a Thermo Finnigan MS. Column: Waters XBridge™ C18, 4.6×50 mm, Mobile phase A: 0.1% formic acid (aq.), Mobile phase B: acetonitrile, Flow: 1 mL/min, Injection volume: 3-20 μL, Detection: 220-320 nm, Gradient: 0% to 100% B in 5 min, buffers A or C were used.

NMR analyses were performed on a Varian Mercury 400 instrument operating at 400 MHz. Residual solvent peak was used as an internal standard.

The assay and purity determination of the compounds were performed by gradient liquid chromatography with UV-detection at 260 nm. That means that a specific volume of the solution was evaporated and the residue was analysed by chromatography and compared with that of a chromatogram of a known amount of said intermediate.

Column: Hypersil Gold C18, 4.6×150 mm, 3 μm (Thermo), Column temperature: 40° C., Column oven: Dionex TCC-3000 SD, Pump: Dionex LPG-3400 SD, Flow rate: 1 mL/min, Injector: Dionex WPS-3000 SL, Injection volume: 10 μL, Detector: Dionex DAD-3000, Wavelength: 260 nm, Data collecting system: Chromeleon.

XRPD data were collected on a Bruker D8 Advance (2005) instrument. Radiation Copper Ka, λ=1.54180 Å, Kb filter 0.020 mm Ni foil, Anode voltage: 40 kV, Anode current 40 mA, Detector: LynxEye (1 D-position sensitive), Slits 0.6 mm and 8 mm, Step size 0.02°, Scan speed 0.2 s/step, Interval (2Θ) (3-35) ° in 2θ scale.

Regular Water Solubility Test

Water solubility tests for the salts as described herein were performed as follows unless otherwise stated. 0.05 g of each salt was weighed in a flask and the mass of flask+salt (m-vs) was recorded. Water was slowly added dropwise into the flask with salt until full dissolution was achieved as observed by the naked eye. The mass of flask+salt+solvent (m-sys) was recorded. The solubility expressed as “grams of solute/kg of solvent”, i.e. “grams of salt/kg of solvent”, was calculated according to the equation:

$$Solubility = \frac{( s ) \times 1000}{( m - s v s ) - ( m - v s )} \qquad Eq . 1$$

In Eq. 1:

(s) stands for the weight of the salt measured in kg,
(m-sys) stands for the mass of the flask+salt+solvent measured in kg, and
(m-vs) stands for the mass of the flask+salt measured in kg.

The value of (s) was 0.05/1000 kg.

Since the solubility was measured in water, and water has a density of 1 g/mL the unit of the solubility may be g/L or mg/mL.

Flask Method Water Solubility Test

In some cases, a further water solubility test (Flask method water solubility test) was performed as follows. An excess of salt was added to water. The mixture was equilibrated (shaking) for at least 24 hours thereby providing a saturated salt solution. Then the saturated solution was clear-filtered and transferred into a clean pre-weighed flask (mv). The mass of flask+saturated solution (mvs) was recorded. The solvent was evaporated under reduced pressure until constant mass. Flask containing dried residue was weighed (mvdr). The solubility expressed as “grams of solute/kg of solvent”, i.e. “grams of salt/kg of solvent”, was calculated according to the equation:

$$Solubility = \frac{( m v d r - m v ) \times 1000}{( m v s - m v d r )} \qquad Eq . 2$$

In Eq. 2:

(mvdr-mv) is the weight difference in kg between (i) the mass of the flask containing dried residue after evaporation of the solvent and (ii) the mass of the flask, and (mvs-mvdr) is the weight difference in kg between (i) the mass of the flask including the saturated salt solution and (ii) the mass of the flask containing the dried residue. Since the solubility was measured in water, and water has a density of 1 g/mL the unit of the solubility may be g/L or mg/mL.

Hygroscopicity Tests

Hygroscopicity test of the L-tartaric salt of [2-(3-fluoro-5-methanesulfonylphenoxy)-ethyl](propyl)amine was performed by keeping exact weight samples of the salt at varied humidity at 30° C. After one week, the samples were weighed again and based on the original weight the percentage weight difference was calculated.

Hygroscopicity test of the hydrochloric acid salt of [2-(3-fluoro-5-methanesulfonyl-phenoxy)ethyl](propyl)amine was recorded on a TA instrument Q550000SA. The temperature was 25° C. using step intervals of 10% between 0% and 95% RH in two consecutive cycles.

### Abbreviations

AIM abnormal involuntary movements
DAT dopamine transporter
g gram(s)
mg milligram(s)
HCl Hydrochloric acid

### EtOH Ethanol

### HPLC High Performance Liquid Chromatography

6-HODA 6-hydroxydopamine
LID levodopa-induced dyskinesia
M molar, i.e. mole(s)/litre
MFB medial forebrain
MTBE Methyl tert-butyl ether
min. minute(s)
mg milligram(s)
mL millilitre
mol mole
mmol(e) millimole

### MS Mass Spectrometry

nM nanoMolar

### NMR Nuclear Magnetic Resonance

i-PrOAc Isopropyl acetate

### THF Tetrahydrofurane

### XRP X-Ray Powder

### XRPD X Ray Powder Diffraction

UV ultraviolet

### A Angstrom

### DVS Dynamic Vapor Sorption

### RH Relative Humidity

b.i.d. Twice (two times) a day

### TA Thermal Analysis

### Example 1

### Synthesis of 2-(3-fluoro-5-methanesulfonylphenoxy)-N-propylacetamide

To a solution of 3-fluoro-5-methanesulfonylphenol (see WO 2006/137790; 20.6 g, 152 mmol) in i-PrOAc (290 mL) was added 2-chloro-N-propylacetamide (29.0 g, 152 mmol) followed by potassium carbonate (42.0 g, 304 mmol). The reaction mixture was heated to reflux temperature and stirred at this temperature for 20 h. The mixture was cooled to room temperature and then water (320 mL) was added. The formed slurry was stirred for 2 h and the precipitate was isolated by filtration. The filter cake was washed with water (2×115 mL) and then with ethanol (3×90 mL). The product was dried by pulling air through it for 4 h. There was obtained 40.0 g (91%) of 2-(3-fluoro-5-methanesulfonylphenoxy)-N-propylacetamide as a solid with a purity of >99 area % (HPLC). ¹H NMR (400 MHz, DMSO-d₆): δ 0.83 (t, 3H), 1.45 (m, 2H), 3.09 (m, 2H), 3.26 (s, 3H), 4.63 (s, 2H), 7.23 (m, 1H), 7.38 (m, 2H), 8.20 (m, 1H).

### Example 2

### Synthesis of [2-(3-fluoro-5-methanesulfonylphenoxy)ethyl](propyl)amine

A mixture of 2-(3-fluoro-5-methanesulfonylphenoxy)-N-propylacetamide (39.0 g, 135 mmol) and THF (390 mL) was heated to 35° C. and 1M solution of BH₃.THF complex in THF (277 mL, 277 mmol) was added over 1 h. The mixture was stirred at 35° C. for 4 h, then at room temperature overnight and then cooled to 7° C. Water (195 mL) was slowly added followed by 37% HCl (6.3 mL, 200 mmol) and the mixture was heated to 56° C. for 3.5 h. Additional amount of water (40 mL) was added followed by 37% HCl (2.5 mL) and the stirring was continued for 28 h at 56° C. After allowing the mixture to cool to room temperature, it was diluted with water (195 mL) and then washed with MTBE (2×200 mL). The pH was adjusted to 11.1 by adding an aqueous solution of NaOH (50%) and then the mixture was extracted with MTBE (2×215 mL). The organic solutions were washed with water (2×120 mL) and then concentrated under reduced pressure until the remaining volume was 60 mL. EtOH (120 mL) was added and the distillation continued until the volume left was approximately 60 mL. The co-evaporation with EtOH was repeated twice and finally the evaporation continued until the remaining volume was approximately 60 mL. The desired [2-(3-fluoro-5-methanesulfonylphenoxy)ethyl]-(propyl)amine was obtained as an EtOH-solution (1.5 M) with a purity of >99 area % (HPLC) and the yield (66%) was determined by using an aliquot evaporation method. ¹H NMR (400 MHz, CDCl₃): δ 0.95 (t, 3H), 1.56 (m, 2H), 2.67 (t, 2H), 3.04 (m, 2H), 3.06 (s, 3H), 4.14 (t, 2H), 6.90 (m, 1H), 7.2-7.3 (m, 2H).

### Example 3

### Synthesis of [2-(3-fluoro-5-methanesulfonylphenoxy)ethyl](propyl)amine hemi-L-tartrate

The EtOH-solution of [2-(3-fluoro-5-methanesulfonylphenoxy)ethyl](propyl)amine from Example 2 (estimated amount: 24.5 g, 88.9 mmol) was diluted with EtOH until the concentration was 0.40 M. A solution of L-tartaric acid (6.81 g, 45.4 mmol) in water (20 mL) was added. The formed slurry was heated to reflux temperature and additional amounts of EtOH (50 mL) and water (5 mL) were added. The heating was continued until all solids dissolved. After allowing the mixture to cool to room temperature, the formed slurry was stirred at room temperature overnight, and then at 5 to 10° C. for 5 h. The precipitate was isolated by filtration and the filter cake was washed with EtOH (3×35 mL).

The product was dried by pulling air through it for 20 min. There was obtained 29.9 g (96%) of [2-(3-fluoro-5-methanesulfonylphenoxy)ethyl](propyl)amine hemi-L-tartrate as a solid with an LC-purity of 99.9%. ¹H NMR (400 MHz, DMSO-d₆): δ 0.89 (t, 3H), 1.55 (m, 2H), 2.74 (t, 2H), 3.13 (t, 2H), 3.28 (s, 3H), 3.89 (s, 1H), 4.27 (t, 2H), 7.25 (m, 1H), 7.3-7.4 (m, 2H).

X-ray powder diffraction analysis was performed on a sample of the crystals of the hemi-L-tartrate salt of [2-(3-fluoro-5-methanesulfonylphenoxy)ethyl](propyl)amine as prepared above according to standard methods using the instrument, equipment and the conditions described in the general description. The analysis provided the diffractogram depicted in FIG. 1. The main characteristic peaks, with positions and relative intensities, have been extracted from the diffractogram in FIG. 1 and is given below in Table 1.

It will be understood that the relative intensities of peaks may vary according to the orientation of the sample under test and on the type and setting of the instrument used so that the intensities in the XRD traces included herein are illustrative and not intended to be used for absolute comparison.

TABLE 1
Positions and intensities of the major peaks in the XRP-diffractogram
of the salt of Formula IVa which is a combination of the compound
of Formula I and L − (+) − tartaric acid in a ratio of 1:0.5.

| Angle 2θ/° | d-value/Å | % Relative intensity |
|---|---|---|
| 7.18 | 12.3 | 9 |
| 11.44 | 7.73 | 1 |
| 11.78 | 7.51 | 3 |
| 12.43 | 7.11 | 49 |
| 13.02 | 6.79 | 100 |
| 13.94 | 6.35 | 2 |
| 14.40 | 6.15 | 71 |
| 15.33 | 5.78 | 19 |
| 16.09 | 5.50 | 5 |
| 18.07 | 4.90 | 41 |
| 18.64 | 4.76 | 21 |
| 19.62 | 4.52 | 41 |
| 19.92 | 4.45 | 46 |
| 20.65 | 4.30 | 4 |
| 21.10 | 4.21 | 46 |
| 21.44 | 4.14 | 36 |
| 21.66 | 4.10 | 32 |
| 22.13 | 4.01 | 21 |
| 22.35 | 3.97 | 20 |
| 22.99 | 3.86 | 10 |
| 23.86 | 3.73 | 15 |
| 24.36 | 3.65 | 50 |
| 24.75 | 3.59 | 17 |
| 25.02 | 3.56 | 3 |
| 25.66 | 3.47 | 10 |
| 26.06 | 3.42 | 4 |
| 26.35 | 3.38 | 10 |
| 27.66 | 3.22 | 13 |
| 28.01 | 3.18 | 1 |
| 28.44 | 3.14 | 8 |
| 29.20 | 3.06 | 1 |
| 30.16 | 2.96 | 4 |
| 30.43 | 2.94 | 2 |
| 30.96 | 2.89 | 20 |
| 31.44 | 2.84 | 8 |
| 32.48 | 2.75 | 7 |
| 33.21 | 2.69 | 2 |
| 33.74 | 2.65 | 2 |
| 34.15 | 2.62 | 2 |
| 34.81 | 2.58 | 5 |

### Examples 4-7: General Procedure for the Synthesis of Various Acid Addition Salts of [2-(3-fluoro-5-methanesulfonylphenoxy)ethyl](propyl)amine

The appropriate acid was added to an EtOH-solution of [2-(3-fluoro-5-methane-sulfonylphenoxy)ethyl](propyl)amine in ethanol and the mixture was heated to reflux until full dissolution, then cooled to room temperature. If precipitation occurred, the resultant solid was collected by filtration. The base/acid ratio of the obtained salts was determined by ¹H NMR spectroscopy with a relaxation time of at least 10 seconds. Melting point was determined by DSC (Differential scanning calorimetry) and the solid-state characterization was determined by XRPD, which was used to determine if the precipitated salt was crystalline.

### Example 4

### Fumaric acid Salt of [2-(3-fluoro-5-methanesulfonylphenoxy)ethyl](propyl)amine

The title salt was prepared according to the general procedure above. The salt was crystalline as determined by XRPD.

Yield: 78%.

Base/acid ratio: 2:1.

Melting point: 184.9° C.

Solubility in water: 92 mg/mL.

XRPD analysis provided the diffractogram in FIG. 2. The main characteristic peaks, with positions and relative intensities, have been extracted from the diffractogram in FIG. 2 and is given below in Table 2.

Table 2: Positions and intensities of the major peaks in the XRP-diffractogram of the salt of Formula Va which is a combination of the compound of Formula I and fumaric acid in a ratio of 1:0.5.

TABLE 2

| Angle 2θ/° | d-value/Å | % Relative intensity |
|---|---|---|
| 7.62 | 11.6 | 42 |
| 11.17 | 7.92 | 1 |
| 12.66 | 6.98 | 4 |
| 12.98 | 6.82 | 12 |
| 13.79 | 6.42 | 1 |
| 15.27 | 5.80 | 100 |
| 15.54 | 5.70 | 6 |
| 16.23 | 5.46 | 1 |
| 16.64 | 5.32 | 1 |
| 18.55 | 4.78 | 11 |
| 18.88 | 4.70 | 2 |
| 19.15 | 4.63 | 2 |
| 19.51 | 4.55 | 2 |
| 19.92 | 4.45 | 4 |
| 21.10 | 4.21 | 2 |
| 21.47 | 4.14 | 2 |
| 21.84 | 4.07 | 12 |
| 22.60 | 3.93 | 5 |
| 22.65 | 3.92 | 8 |
| 22.98 | 3.87 | 22 |
| 23.47 | 3.79 | 1 |
| 24.08 | 3.69 | 9 |
| 24.89 | 3.57 | 1 |
| 25.34 | 3.51 | 2 |
| 25.78 | 3.45 | 3 |
| 26.01 | 3.42 | 3 |
| 27.47 | 3.24 | 1 |
| 28.62 | 3.12 | 1 |
| 29.33 | 3.04 | 1 |
| 29.52 | 3.02 | 1 |
| 30.79 | 2.90 | 7 |
| 31.40 | 2.85 | 3 |
| 32.83 | 2.73 | 1 |
| 33.11 | 2.70 | 1 |
| 34.00 | 2.64 | 1 |

### Example 5

### Maleic Acid Salt of [2-(3-fluoro-5-methanesulfonylphenoxy)ethyl](propyl)amine

The title salt was prepared according to the general procedure above. The salt was crystalline as determined by XRPD.

Yield: 88%.

Base/acid ratio: 1:1.

Melting point: 141.5° C.

Solubility in water: 35 mg/mL.

### Example 6

### Succinic Acid Salt of [2-(3-fluoro-5-methanesulfonylphenoxy)ethyl](propyl)amine

The title salt was prepared according to the general procedure above with the exception that the solution was cooled to −18° C. until precipitation occurred. The salt was crystalline as determined by XRPD.

Yield: 75%.

Base/acid ratio: 1:1.

Melting point: 109.2° C.

Solubility in water: 285 mg/mL.

### Example 7

### L-tartaric Acid Salt of [2-(3-fluoro-5-methanesulfonylphenoxy)ethyl](propyl)amine

The title salt was prepared according to the general procedure above. The salt was crystalline as determined by XRPD.

Yield: 73%.

Base/acid ratio: 2:1.

Melting point: 187.6° C.

Solubility in water (regular water solubility test): 185 mg/mL.

Solubility in water (flask method water solubility test): 252.6 mg/mL.

### Comparative Example (Water Solubility)

In comparative tests for the determination of the water solubility for the hydrochloric acid salt of [2-(3-fluoro-5-methanesulfonylphenoxy)-ethyl](propyl)amine it was concluded that said hydrochloric acid salt (i.e. Example 1 of WO 2012/143337) had a water solubility of 197 mg/mL (regular water solubility test) and 270 mg/mL (flask method water solubility test), respectively.

### Hygroscopicity Determination of the L-tartaric Acid Salt of [2-(3-fluoro-5-methanesulfonylphenoxy)ethyl](propyl)amine According to Example 7

TABLE 3

| | Weight |
|---|---|
| Relative | difference (%) |
| Humidity (%) | Example 7 |
| 0 | −0.26 |
| 6 | 0.00 |
| 11 | −0.06 |
| 22 | 0.12 |
| 33 | −0.18 |
| 45 | −0.13 |
| 56 | −0.15 |
| 73 | −0.24 |
| 75 | −0.07 |
| 83 | 0.06 |
| 97 | −0.08 |

As shown in Table 3, the L-tartaric acid salt of [2-(3-fluoro-5-methanesulfonylphenoxy)ethyl](propyl)amine according to Example 7 does not adsorb or desorb any significant amount of water at any humidity. Thus, the salt has a very low hygroscopicity, i.e. a weight change of ±0.3% or less, even when it is exposed to a very high relative humidity such as 73%, 75%, 83% or 97% for 7 days at 30° C.

### Comparative Example (Hygroscopicity)

In another test utilizing the DVS technique for the determination of the hygroscopicity for the hydrochloric acid salt of [2-(3-fluoro-5-methanesulfonylphenoxy)-ethyl](propyl)amine it was concluded that said hydrochloric acid salt (i.e. Example 1 of WO 2012/143337) gained approximately 3% of weight at 95% relative humidity and 25° C. The cycles were repeatable.

Remarks

As can be seen in the Examples above, the maleic acid salt has a low solubility and a relatively low melting point whereas the succinic acid salt has a very good solubility but a low melting point. On the other hand, the fumaric acid salt, and in particular, the L-tartaric acid salt have high melting points as well as high solubilities in water. Also, even though the hydrochloric acid salt according to Example 1 of WO 2012/143337 has a high melting point and high water solubility, it is hygroscopic at high relative humidities. The L-tartaric acid salt according to the present disclosure is not hygroscopic at any relative humidities. These beneficial physical properties taken together make the fumaric acid salt and in particular the L-tartaric acid salt of [2-(3-fluoro-5-methanesulfonyl-phenoxy)ethyl](propyl)amine very good drug candidates when it comes to possessing pharmaceutical properties, i.e. properties making it suitable for use as a pharmaceutical, such as handling and/or storage properties.

### Example 8

### Human Clinical Study in Parkinson's Patients with L-DOPA Induced Dyskinesia (LIDs) Using the L-tartaric Acid Salt of (2-(3-fluoro-5-methanesulfonyl-phenoxy)ethyl](Propyl)amine of Example 7

Home diaries have gained wide acceptance as endpoints for clinical development of therapeutics aiming to reduce treatment-related motor complications. Motor fluctuations are associated with compromise in activities of daily living and health-related quality of life. In clinical trials performed by Integrative Research Laboratories Sweden AB patients record their 24-hours motor function in 30-minute intervals, beginning at midnight. For each 30-minute interval the patient rates the state he or she has been in for the past 30 minutes; OFF, ON without troublesome dyskinesia or ON with troublesome dyskinesia. The patient also denoted the time when he or she has been asleep. It has been demonstrated that OFF-time and ON-time with troublesome dyskinesia are generally considered by patients to be “bad time” with regard to motor function, whereas ON-time without dyskinesia and ON-time without troublesome dyskinesia are generally considered to be “good ON-time”.

In general, an “OFF” time reduction or “good ON-time” increase of 1 hour may be considered clinically significant and has been used as an assumption in power calculations in clinical trials. Therefore, it can be assumed that shift towards more “good ON-time” of a minimum of 1-hour daily represents a clinically meaningful effect, considering the total time spent in the daily ON state (ON with and without troublesome dyskinesia) is not negatively affected by the treatment.

For inclusion in the clinical trial, patients must demonstrate ability to complete the 24-hour patient home diaries. A valid diary is defined as not having more than 2 hours of invalid data entries (4 invalid entries) over a given 24-hour period. An invalid diary entry is defined as more than one entry recorded in each half-hour interval, an unreadable entry, or the absence of an entry in each half-hour interval. The average diary information from 3 valid diaries (if available) for each visit will be used to calculate diary-based efficacy endpoints. If there are only 2 valid diaries for a visit, then the average information from the 2 valid diaries will be used. If only one diary is valid, information from the single valid diary will be used. If no valid diaries were available for a patient visit, then the diary information was considered missing.

Method

At run-in and following treatment patients were asked to complete home diaries describing their motor status in 30-minute intervals for 24 hours. Patients were asked to describe their past 30-minute motor status in one of four categories: Asleep, OFF, ON, or ON with troubling dyskinesia. Descriptions of each category were included in the diary as follows: ON: Good or practically normal mobility.

ON with troubling dyskinesia: Troubled by involuntary twisting, turning movements. These movements are different from the rhythmic “tremor” which is a symptom of Parkinson's Disease itself.

OFF: Stiffness, marked decrease in mobility, or immobility.

Asleep: Time spent asleep.

### Results and Conclusion

It has been found in human studies that through reduction in troublesome dyskinesias (LIDs) in patients suffering from Parkinson's Disease treated with L-DOPA (levodopa) resulted in more daily hours of good mobility (good ON time) at plasma concentrations of 50-200 nM of the drug measured 2 hours after administration of drug in the morning.

This plasma concentration range was obtained by administering to patients 2.5 mg b.i.d. −10 mg b.i.d. of the L-tartaric acid salt of [2-(3-fluoro-5-methanesulfonyl-phenoxy)ethyl](propyl)amine. The doses (2.5 mg-10 mg) are calculated on the non-salt form of [2-(3-fluoro-5-methanesulfonyl-phenoxy)ethyl](propyl)amine.

Table 4. Improvement in Good ON time (i.e. the time the patient indicated as “ON” meaning good or practically normal mobility) in Parkinson's patients with L-DOPA induced dyskinesia (LIDs) using the L-tartaric acid salt of [2-(3-fluoro-5-methanesulfonyl-phenoxy)ethyl](propyl)amine according to Example 7 or placebo.

TABLE 4

| Dose treatment | Improvement in good | Number |
|---|---|---|
| twice-daily (b.i.d.) | ON time (mean values) | of patients |
| 5 mg or less (2.5 mg | 2.1 h | 9 |
| in some patients) | | |
| 7.5 mg | 5.6 h | 8 |
| 10 mg or more (12.5 mg | 1.4 h | 20 |
| in some patients) | | |
| Placebo | 1.0 | 34 |

It was concluded that administration of [2-(3-fluoro-5-methanesulfonyl-phenoxy)ethyl](propyl)amine in an amount from 2.0 mg up to 10 mg such as 2.5 mg, 5.0 mg or 7.5 mg to a patient suffering from Parkinson's disease resulted through reduction in troublesome dyskinesia (LIDs) in more daily hours of good mobility (good ON time). Further, it was concluded that administration of [2-(3-fluoro-5-methanesulfonyl-phenoxy)ethyl](propyl)amine in an amount from 2.0 mg up to 10 mg such as 2.5 mg, 5.0 mg or 7.5 mg increased the good ON time to a larger extent than administration of [2-(3-fluoro-5-methanesulfonyl-phenoxy)ethyl](propyl)amine in an amount equal to or above 10 mg.

### Example 9

### Study in Rodent Model of L-DOPA Sensitization Using the L-tartaric Acid Salt of [2-(3-fluoro-5-methanesulfonyl-phenoxy)ethyl](propyl)amine of Example 7

The unilateral 6-OHDA lesion model is an established animal model of Parkinson's disease (see e.g. Exp. Neurol. 194: 66-75 (2005)). The neurotoxin 6-OH-DA is injected in the medial forebrain bundle (MFB), resulting in depletion of dopamine e.g. in the striatum. Upon repeated administration of L-DOPA, the animals with such a lesion display sensitization to L-DOPA, captured by rotational behaviour measured as associated with the development of LIDs. The effects of IRL790 (as a HCl-salt) on fully established AIMs have been published previously (J. Pharmacol. Exp. Ther. 374:113-125 (2020)). Thus, in this article the administration of IRL790 was preceded by administration of L-DOPA. In the present study the effect of the L-tartaric acid salt of [2-(3-fluoro-5-methanesulfonyl-phenoxy)ethyl](propyl)amine of Example 7 when co-administered with L-DOPA throughout the study was investigated. The co-administration of said test compound with the L-DOPA was not preceded by treatment with L-DOPA. The clinically used antidyskinetic compound amantadine (as a HCl-salt) together with L-DOPA was used for comparison.

Methods

Animals

For the evaluation of sensitization to L-DOPA the rodent unilateral 6-OH-DA-lesion model was used. Adult 150˜200 g female Sprague Dawley rats (Charles River) were housed in temperature- and humidity-controlled rooms (20° C., 53% humidity) with a 12 h dark/light cycle. The rats had access to standard food pellets and water ad libitum. The experiments were approved by the local ethical committee at Karolinska Institute (Stockholm North Ethical Committee, N1525/2017) and conducted in accordance with the European Communities Council Directive of 24 Nov. 1986 (86/609/EEC).

6-OHDA Lesioning

Experimental procedures including unilateral 6-OH-DA-lesions and behavioral assessments were carried as follows. Rats were anesthetized using a mixture of ketamine (100 mg/kg, i.p.; Parke-Davis, Boxmeer, The Netherlands) and xylazine (10 mg/kg, i.p.; Bayer, Kiel, Germany), pretreated with a mixture of desipramine (25 mg/kg, i.p.; Sigma-Aldrich) and pargyline (5 mg/kg, i.p.; Sigma-Aldrich) 30 minutes before lesioning to prevent uptake of 6-OHDA into noradrenergic neurons, and to prevent extracellular metabolism of 6-OHDA, respectively. Rats were placed in a stereotaxic frame (David Kopf Instruments, KOPF®, USA) and injected with temgesic (0.1 mg/kg, s.c., Apoteket, Sweden) before surgery; 2 μl 6-OHDA (5 μg/μl in saline containing 0.02% w/v ascorbic acid, Sigma-Aldrich) was injected into the right MFB, stereotaxic coordinates AP: −2.5 mm, ML: −2.0 mm, DV: −8.5 mm vs. bregma and dural surface (The Rat Brain in Stereotaxic Coordinates 6^{th} Edition, George Paxinos and Charles Watson, 2007) in order to selectively lesion dopaminergic cells in the right brain hemisphere. Post-operative analgesia (Temgesic, s.c.) was administered twice within 48 hours after surgery. Two weeks following surgery, rats were treated with apomorphine (1 mg/kg i.p.; Sigma-Aldrich) to verify successful lesioning. Only the rats rotating more than 100 contralateral turns during a 30 min period were used for subsequent experiments. Four weeks after surgery, rotation test was done with treatments of saline and test compounds. The efficacy of the 6-OHDA lesion was later examined by using dopamine transporter (DAT) autoradiography. Only when DAT binding was reduced more than 90% by 6-OHDA, successful lesioning was indicated.

Treatment with Test Compounds

Whenever L-DOPA was used in this study, it was administered together with benzerazid HCl-salt 7.5 mg/kg i.p., which is a standard procedure when L-DOPA is administered to rats in the unilateral 6-OHDA lesion model. Chronic treatment of four groups of rats was started with either: (1) L-DOPA (10 mg/kg), (2) [2-(3-fluoro-5-methanesulfonyl-phenoxy)ethyl](propyl)amine tartaric acid salt (3 mg/kg s.c.), (3) L-DOPA (10 mg/kg)+[2-(3-fluoro-5-methanesulfonyl-phenoxy)ethyl](propyl)amine tartaric acid salt (3 mg/kg s.c.), or (4) the corresponding vehicle (saline, i.p.) given once daily for 2 weeks. Additionally, two groups of rats were treated with either amantadine HCl salt (40 mg/kg ip), or L-DOPA (10 mg/kg)+amantadine HCl salt (40 mg/kg i.p.), once daily for 14 days. Behavioural assessments were performed on Day 1, Day 7, and Day 14 of chronic treatment. Thus, on Days 1, 7 and 14 contralateral rotations (i.e. rotations in the opposite direction to the side of the 6-OH-DA-lesion) were measured. Contralateral rotations were measured by Rotorat over 2.5 hours after the administration of test compound. On Day 15, 30 minutes following the last injection of test compounds, animals were sacrificed. The MFB lesion was verified by dopamine transporter (DAT) binding autoradiography.

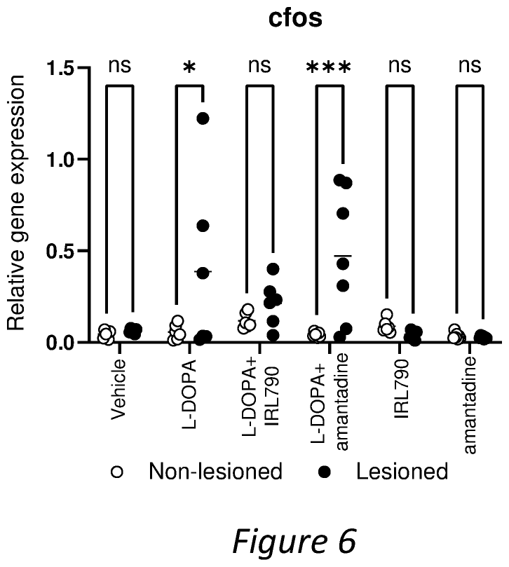

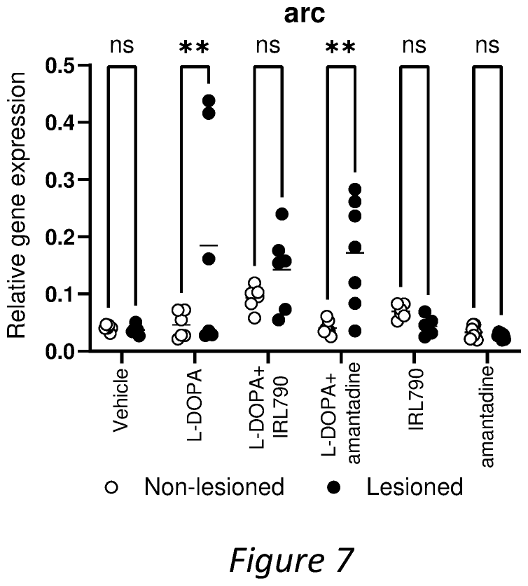

Striatal gene expression analysis of a selected panel of genes was performed by qPCR on the dissected tissue (see for instance J. Pharmacol. Exp. Ther. 374, 113-125 (2020)). Results for two genes, cfos and arc, are shown in FIGS. 6 and 7. Similar results (not shown) were observed for the remaining genes in the panel (nptx2, egr, npas4, pdyn, homer).

Rotation Test by ROTORAT

The rats were put into the chambers prior to starting the recording. Placed the harness (ENV-500JM, MED Associates, Inc®, USA) on the rats and attached the harness to the velcro at the end of the swivel. Placed the rats in the chamber and reattached the swivel to the chamber, and started recording with the software ROTORAT (SOF-801, MED Associates, Inc®, USA). The rotational movement was displayed in real time, the Full (revolutions of 360 degrees, without changing direction) of counterclockwise was used for data analysis. At the end of the trials, the recordings were automatically stopped.

The behavioural data, namely number of contralateral rotations were analysed by means of two-way analysis of variance (ANOVA) followed by post-hoc Fisher's Least Significant Difference (LSD) test, assessing the effects of time, treatment, and treatment*time interactions.

Results

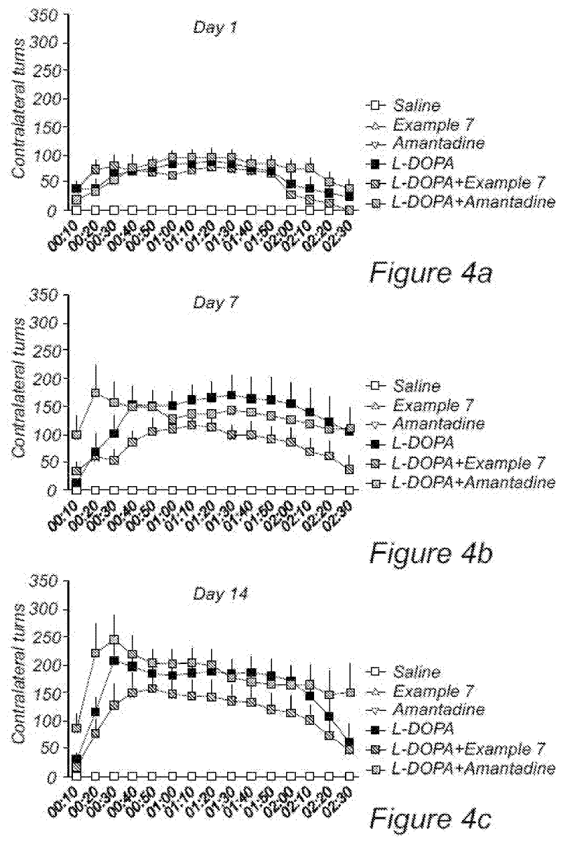

The sensitization to repeated dosage of L-DOPA, as measured by contralateral turns following administration of Saline, L-DOPA, [2-(3-fluoro-5-methanesulfonyl-phenoxy)ethyl](propyl)amine tartaric acid salt, L-DOPA+[2-(3-fluoro-5-methanesulfonyl-phenoxy)ethyl](propyl)amine tartaric acid salt, amantadine HCl salt, or L-DOPA+amantadine HCl salt on Day 1, Day 7, and Day 14, is shown in FIGS. 4a, 4b and 4c. Shown are means±SEM of number of contralateral turns measured during each 10 minute interval of the recording session. N=6-8/group.

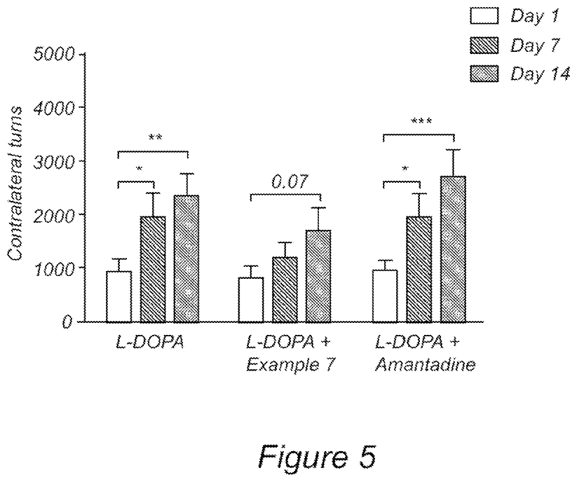

Contralateral turns were only observed in rats that received L-DOPA. The groups receiving saline, amantadine HCl salt only, or [2-(3-fluoro-5-methanesulfonyl-phenoxy)ethyl](propyl)amine tartaric salt displayed no contralateral turns. Thus, in FIGS. 4a, 4b and 4c, the contralateral turns for test compounds [2-(3-fluoro-5-methanesulfonyl-phenoxy)ethyl](propyl)amine tartaric salt (Example 7) and amantadine cannot be seen because these values are in the baseline together with the contralateral turns for saline. The L-DOPA group displayed increasing contralateral rotational behavior over the three time-points assessed. This was confirmed with the statistical analysis, showing significant differences Day 7 vs Day 1 and Day 14 vs Day 1, in the L-DOPA group (FIG. 5). More specifically, FIG. 5 shows total number of contralateral turns in rats chronically treated with L-DOPA, L-DOPA+[2-(3-fluoro-5-methanesulfonyl-phenoxy)ethyl](propyl)amine tartaric acid salt, and L-DOPA+amantadine HCl salt for 2 weeks. N=6-8/group. Stars denote statistical significance of difference between the Day 1, Day 7, and Day 14 assessments (2-way ANOVA followed by Fisher's LSD) * p<0.05, ** p<0.01, *** p<0.001. A similar response pattern was evident for the L-DOPA+amantadine HCl salt group (FIGS. 4a-4c, FIG. 5). In contrast, rats co-treated with L-DOPA+[2-(3-fluoro-5-methanesulfonyl-phenoxy)ethyl](propyl)amine tartaric acid salt displayed a less pronounced increase over time, and there was no significant difference in rotational behavior on Day 7 or Day 14, vs Day 1 (FIG. 4a-4c, FIG. 5)).

The gene expression data are consistent with the results of the behavioral (ROTORAT) tests. Striatal expression of immediate early response genes (IEGs) cfos and arc, which may be used as an index of sensitization, were significantly increased by L-DOPA treatment. These increases were attenuated in animals receiving IRL790 (the salt of Example 7) but not in those receiving amantadine (shown in FIGS. 6 and 7). Similar trends (not shown) were also observed for further IEGs (nptx2, egr, npas4, pdyn, homer).

### CONCLUSION

The present study using the unilateral 6-OH-DA model, which is a validated model of L-DOPA induced dyskinesias (LIDs) in Parkinson's disease, showed that [2-(3-fluoro-5-methanesulfonyl-phenoxy)ethyl](propyl)amine tartaric acid salt when co-administered with L-DOPA did not provide L-DOPA sensitization as evidenced by the small and not statistically significant increase in contralateral turns on Days 7 and 14, compared to Day 1 of repeated administration of L-DOPA. The contralateral turning behaviour reflects an increased sensitivity to dopamine receptor stimulation on the lesioned side, which develops over time upon chronic dosing with L-DOPA. This L-DOPA sensitization process is an important aspect of the development of AlMs, i.e. the involuntary movements specifically representing LIDs (see e.g. Exp Neurol. 1998 June; 151(2):334-42). Hence, the lack of L-DOPA sensitization strongly suggests a disease modifying effect of [2-(3-fluoro-5-methanesulfonyl-phenoxy)ethyl](propyl)amine such as its tartaric acid salt, i.e. that [2-(3-fluoro-5-methanesulfonyl-phenoxy)ethyl](propyl)amine or its tartaric acid salt blocks the pathophysiological mechanisms underlying LIDs. This is a finding that is corroborated by gene expression data, showing a dampening of the L-DOPA-induced upregulation of IEG response by IRL790 (the salt of Example 7). Importantly, the reference compound amantadine HCl salt, which is the only compound approved for the treatment of LIDS at present, did not display such effects on the L-DOPA sensitization, neither in terms of dyskinetic behavior nor gene expression. Both [2-(3-fluoro-5-methanesulfonyl-phenoxy)ethyl](propyl)amine and amantadine are known to decrease AIMs as such upon acute administration, in the unilateral 6-OHDA model. Of note, both compounds have also been observed to reduce LI Ds in clinical studies, reflecting the validity of the model. These results clearly demonstrate that the ability to minimize or avoid L-DOPA sensitization is displayed by the combination of L-DOPA and [2-(3-fluoro-5-methanesulfonyl-phenoxy)ethyl](propyl)amine such as its tartaric acid salt, but not the combination of amantadine HCl salt and L-DOPA. This shows that [2-(3-fluoro-5-methanesulfonyl-phenoxy)ethyl](propyl)amine such as its tartaric acid salt, in addition to its acute, specific, antidyskinetic effects on ongoing dyskinesias as previously published, also can slow down or halt the maladaptive changes in the brain's responsiveness to L-DOPA, which is believed to underlie the development of LI Ds, i.e. it should be regarded as disease modifying. Such a disease modifying treatment is a great advantage since the development of LIDs is prevented instead of just waiting for the LIDs to occur and then treating them. Further, it was concluded that a combination of L-DOPA and [2-(3-fluoro-5-methanesulfonyl-phenoxy)ethyl](propyl)amine such as its tartaric acid salt can be used for treating Parkinson's disease without or substantially without development of LIDs.

### REFERENCES

1. New Engl. J. Med. 351: 2498-2508 (2004)
2. WO 2012/143337
3. PCT/EP2020/064046
4. J. Pharmacol. Exp. Ther. 374:113-125 (2020)
5. WO 2020/110128 A1
6. npj Parkinson's Disease. 33: 1-6 (2018)
7. Exp. Neurol. 194: 66-75 (2005)
8. The Rat Brain in Stereotaxic Coordinates 6th Edition, George Paxinos and Charles Watson, 2007
9. Exp Neurol. June; 151(2):334-42 (1998)

1. A method for preventing or reducing sensitization to a pharmaceutical drug used in the treatment of Parkinson's disease, the method comprising administering to a subject in need thereof a therapeutically effective amount of a compound of Formula I 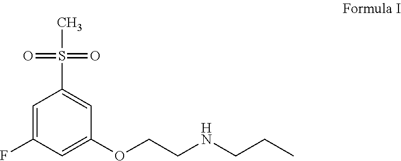 [SMILES: CCCNCCOc1cc(F)cc(S(C)(=O)=O)c1]
or a pharmaceutically acceptable salt thereof.

2. The method of claim 1, wherein the pharmaceutical drug for Parkinson's disease is apomorphine, L-DOPA, a derivative of L-DOPA, pramipexole, ropinirole, or rotigotine, or a pharmaceutically acceptable salt of any one of the foregoing.

3. The method of claim 1, wherein the pharmaceutical drug for Parkinson's disease comprises L-DOPA or a pharmaceutically acceptable salt thereof.

4. The method of claim 1, wherein L-DOPA sensitization is prevented or reduced.

5. The method of claim 1, wherein, prior to being administered the compound of Formula I or a pharmaceutically acceptable salt thereof, the subject has not experienced dyskinesias.

6. The method of claim 1, wherein prior to being administered the compound of Formula I or a pharmaceutically acceptable salt thereof, the subject has not experienced L-DOPA-induced dyskinesias.

7. The method of claim 5, wherein the subject has been treated with a pharmaceutical drug for Parkinson's disease for at least 1 day prior to an initial administration of the compound of Formula I or pharmaceutically acceptable salt thereof.

8. The method of claim 6, wherein the subject has been treated with L-DOPA or a pharmaceutically acceptable salt thereof for at least 1 day prior to an initial administration of the compound of Formula I or pharmaceutically acceptable salt thereof.

9. The method of claim 1, wherein the subject has not previously been administered a pharmaceutical drug for Parkinson's disease.

10. The method of claim 9, wherein the pharmaceutical drug for Parkinson's disease is L-DOPA or a pharmaceutically acceptable salt thereof.

11. The method of claim 9, wherein the compound of Formula I or pharmaceutically acceptable salt thereof is administered to the subject at least 1 day prior to the initial administration of the pharmaceutical drug for Parkinson's disease to the subject.

12. The method of claim 10, wherein the compound of Formula I or pharmaceutically acceptable salt thereof is administered to the subject at least 1 day prior to the initial administration of L-DOPA or a pharmaceutically acceptable salt thereof to the subject.

13. The method of claim 1, wherein the compound of Formula I or pharmaceutically acceptable salt thereof is administered to the subject after the subject has been diagnosed with Parkinson's disease.

14. The method of claim 1, wherein the subject has been diagnosed as being at risk for Parkinson's disease.

15. The method of claim 1, wherein the subject is administered the compound of Formula I or pharmaceutically acceptable salt thereof in an amount corresponding to about 2.0 mg to about 10.0 mg of the compound of Formula I.

16. The method of claim 1, wherein the subject is administered one or more doses of the compound of Formula I or a pharmaceutically acceptable salt thereof in a first amount and then administered one or more doses of the compound of Formula I or a pharmaceutically acceptable salt thereof in a second amount, wherein the second amount is lower than the first amount.

17. The method of claim 16, wherein the first amount corresponds to an amount of about 7.5 up to about 10.0 mg of the compound of Formula I.

18. The method of claim 16, wherein the second amount corresponds to an amount of about 2.5 to about 5.0 mg of the compound of Formula I.

19. The method of claim 1, wherein the compound of Formula I or pharmaceutically acceptable salt thereof is administered to the subject twice per day.

20. The method of claim 1, wherein the compound of Formula I or pharmaceutically acceptable salt thereof is administered to the subject in a total daily dosage corresponding to about 4.0 up to about 20.0 mg of the compound of Formula I.

21. The method of claim 1, wherein the compound of Formula I or pharmaceutically acceptable salt thereof is administered to the subject in a total daily dosage corresponding to about 5.0 to about 15.0 mg of the compound of Formula I.

22. The method of claim 1, wherein the pharmaceutically acceptable salt is a salt of Formula III: 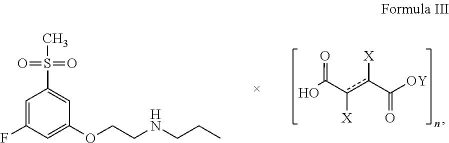
wherein the salt is a combination of a compound of Formula I and an acid of Formula II: 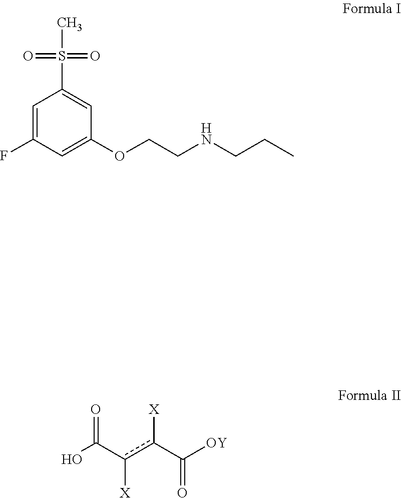
in a ratio of 1:n, wherein:
X is H or OH;
Y is H or a cation selected from Li, Na and K; [symbol]
is a single bond or a double bond; and
n is 0.5 or 1.

23. The method of claim 1, wherein the pharmaceutically acceptable salt is a salt of Formula IV: 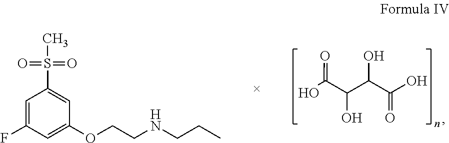
wherein n is 0.5 or 1.

24. The method of claim 22, wherein n is 0.5.

25. The method of claim 23, wherein n is 0.5.

26. A method for optimizing the dosage of a pharmaceutical drug for Parkinson's disease, the method comprising administering to a subject in need thereof a therapeutically effective amount of a compound of Formula I 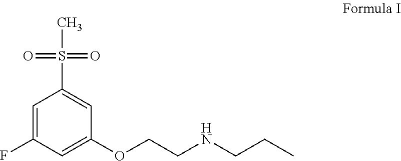 [SMILES: CCCNCCOc1cc(F)cc(S(C)(=O)=O)c1]
or a pharmaceutically acceptable salt thereof.

27-29. (canceled)
